SEQUENCE LISTING (15 sequences)

SEQ ID NO: 1 - protein, 347 aa, Arabidopsis thaliana
MEKPESEKKSAVSDVGAWAMNVISSVGIIMANKQLMSSSGFGFGFATTLTGFHFAFTALVGMVSNATGLSASKHVPLWELLWFSIVANISIAAMNFSLMLNSVGFYQISKLSMIPVVCVLEWILHSKHYCKEVKASVMVVVIGVGICTVTDVKVNAKGFICACTAVFSTSLQQISIGSLQKKYSVGSFELLSKTAPIQAISLLICGPFVDYLLSGKFISTYQMTYGAIFCILLSCALAVFCNISQYLCIGRFSATSFQVLGHMKTVCVLTLGWLLFDSEMTFKNIAGMAIAIVGMVIYSWAVDIEKQRNAKSTPHGKHSMTEDEIKLLKEGVEHIDLKDVELGDTKP

SEQ ID NO: 2 - protein, 357 aa, Arabidopsis thaliana
MSEGQKFQLGTIGALSLSVVSSVSIVICNKALISTLGFTFATTLTSWHLLVTFCSLHVALWMKMFEHKPFDPRAVMGFGILNGISIGLLNLSLGFNSVGFYQMTKLAIIPCTVLLETLFFRKKFSRKIQFSLTILLLGVGIATVTDLQLNMLGSVLSLLAVVTTCVAQIMTNTIQKKFKVSSTQLLYQSCPYQAITLFVTGPFLDGLLTNQNVFAFKYTSQVVFFIVLSCLISVSVNFSTFLVIGKTSPVTYQVLGHLKTCLVLAFGYVLLRDPFDWRNILGILVAVIGMVVYSYYCSIETQQKASETSTQLPQMKESEKDPLIAAENGSGVLSDGGGGVQQKTVAPVWNSNKDFQA

SEQ ID NO: 3 - protein, 353 aa, Arabidopsis thaliana
MSDAQKFQLGTIGALSLSVVSSVSIVICNKALISTLGFTFATTLTSWHLLVTFCSLHVALWMKFFEHKPFDPRAVLGFGVLNGISIGLLNLSLGFNSVGFYQMTKLAIIPCTVVLETIFFRKMFSRKIQFSLVILLLGVGIATVTDLQLNMLGSVLSLLAVITTCVAQIMTNTIQKKYKVSSTQLLYQSCPYQAITLFVTGPFLDGLLTNQNVFAFKYTSQVVFFIVLSCLISVSVNFSTFLVIGKTSPVTYQVLGHLKTCLVLAFGYLLLKDAFSWRNILGILVAVIGMVLYSYYCTLETQQKATETSTQLPQMDENEKDPLVSAENGSGLISDNGVQKQDPVWNSNKDFQA

SEQ ID NO: 4 - protein, 342 aa, Arabidopsis thaliana
MGEMKSMQMGVIGALFLSVASSVSIVICNKALMTNLGFPFATTLTSWHLMVTYCTLHVAYKLNFFENKPIDMRTVVLFGLLNGISIGLLNLSLGFNSIGFYQMTKLAIIPFTVLLETLFLNKKFSQKIKFSLFLLLVGVGIASITDLQLNFVGSVLSLLAIATTCVGQILTNTIQKRLNVTSTQLLYQSAPFQAAILFVSGPFVDKYLTSLNVFSFHYSPIVVGFITLSCLIAVSVNFSTFLVIGKTSPVTYQVLGHLKTCLVLAFGYTLLHDPFTPRNIAGILIAVLGMLLYSYFCSVASKSKQASSDSTFLGKDRDTTPLLGQENENHHEAKKLDKHSPV

SEQ ID NO: 5 - protein, 335 aa, Arabidopsis thaliana
MAPVKKSDKKATLDAAAWMFNVVTSVGIIIVNKALMATYGFSFATTLTGLHFATTTLMTLVLRCLGYIQPSHLPFTELLKFILFANFSIVGMNVSLMWNSVGFYQIAKLSMIPVSCLLEVVFDKIRYSRDTKLSIGLVLVGVGVCTVTDVSVNTKGFVAAFVAVWSTALQQYYVHYLQRKYSLSSFNLLGHTAPAQAATLLIVGPFLDYWLTDKRVDMYDYNSVSVMFITLSCTIAIGTNLSQFICIGRFTAVSFQVLGHMKTILVLVMGFFFFDRDGLNLHVILGMIIAVLGMIWYGNASSKPGGKEKKNYSLPTTRQQKLGATSDSDEHRDKA

SEQ ID NO: 6 - protein, 335 aa, Arabidopsis thaliana
MAPVSKADKKAAVDAAAWMFNVVTSVGIIIVNKALMATYGYSFATTLTGLHFATTTLMTLVLRCLGYIQPSHLPFTELLRFILFANFSIVGMNVSLMWNSVGFYQIAKLSMIPVSCLLEMVFDKIRYSRDTKLSIGLVLVGVGVCTVTDVSVNTKGFVAAFVAVWSTALQQYYVHYLQRKYSLNSFNLLGHTAPAQAATLLVVGPFLDYWLTEKRVDMYDYNLVSVLFITLSCTIAIGTNLSQFICIGRFTAVSFQVLGHMKTILVLIMGFFFFGREGLNLHVVVGMIIAVLGMIWYGNASSKPGGKERRNYSLPTTRQQKLGAASDSDDNEDKA

SEQ ID NO: 7 - protein, 337 aa, Arabidopsis thaliana
MFKKMSATTSKTDQKAALDIASWLFNVVTSVGIILVNKALMATYGFSFATTLTGLHFGTTTLLTTFLTWLGYIQPSQLPWPDLLKFVLFANFSIVGMNVSLMWNSVGFYQIAKLSMIPVSCLLEVMLDNVRYSRDTKLSILLVLAGVAVCTVTDVSVNLNGFLAAAIAVWSTALQQYYVHYLQRKYSLGSFNLLAHTAPVQAASLLLVGPFLDYWLTNQRVDAYNFSFVSLFFLILSCSIAVGTNLSQFICIGRFTAVSFQVLGHMKTILVLVLGFTFFGKEGLNLQVVLGMLIAILGMIWYGNASSKPGGKERRSLSIPITKSQKLSETNESDEKV

SEQ ID NO: 8 - protein, 347 aa, Arabidopsis thaliana; identical to SEQ ID NO: 1

SEQ ID NO: 9 - protein, 348 aa, Arabidopsis thaliana
MEKAENEKKPSAVSDVGAWAMNVTSSVGIIMANKQLMSSSGFGFSFATTLTGFHFALTALVGMVSNATGLSASKHVPLWELLWFSLVANISIAAMNFSLMLNSVGFYQISKLSMIPVVCVMEWVLHSKHYSREVKASVMVVVVGVGICTVTDVKVNAKGFICACTAVFSTSLQQISIGSLQKKYSIGSFELLSKTAPIQAISLLIFGPFVDYFLSGRFISTYKMTYSAMLCILLSCALAVFCNISQYLCIGRFSATSFQVLGHMKTVCVLTLGWLIFDSEMTFKNIAGMVLAVVGMVIYSWAVELEKQRKSKVIPHGKHSMTEDEIKLLKEGIEHMDLKDMELGNNKA

SEQ ID NO: 10 - protein, 283 aa, Arabidopsis thaliana
MAAENERKSPSAVSDMGAWAMNVISSVGIIMANKQLMSSSGFAFSFATTLTGFHFALTALVGMVSNATGFSASKHVPMWELIWFSIVANVSIAAMNFSLMLNSVGFYQLIGSLQKKYSIGSFELLSKTAPIQAFSLLVVGPLVDYLLSGKFIMKYNMSSGCFLFILLSCGLAVFCNISQYLCIGRFSAVSFQVIGHMKTVCILTLGWLLFDSAMTFKNVAGMIVAIVGMVIYSWAMELEKQSIIAAKALNSVKHSLTEEEFELLKEGVETTQSKDVELGRTKD

SEQ ID NO: 11 - protein, 398 aa, Arabidopsis thaliana
MGLVDSLLGKDARKFLKRKDSDAAEAGRALEELRSSLYNELKTSEGAKRQQQRFCGPVVAMSFNFVVAVGIILANKLVMGRVGFNFPIFLTLIHYTVAWILLAFFKSLSLLPMSPPSKTTPFSSLFSLGAVMAFASGLANTSLKHNSVGFYQMAKIAVTPTIVLAEFVLFKKTISSTKVMALAVVSLGVAIATVTDLEFNLFGALVAVAWIIPSAINKILWSNLQQQANWTALALMWKTTPFTVFFLLALMPWLDPPGVLLFKWDLTNSSAILISALLGFLLQWSGALALGATSATSHVVLGQFKTCVILLGGYVIFGSDPGFISICGAIAALGGMSVYTWLNLPGKSIDHMSNKQLPKQNVTVSKPKAEADDGGGETGVTVVSVDPLLSKTITANIV

SEQ ID NO: 12 - protein, 390 aa, Arabidopsis thaliana
MLLTPKMFSSWLRQDVKKILKRKDSDAGERGKALEDLRASLFNRFRSPETPKRQQQQQHRICGPTVALTFNFVVAISIIFMNKWVLKNIGFEFPVFLTFIHYIVAYLLMALLKSFSLLPASPPSTKSSLLPLYTLGIVMSLSTGLANVSLKYNSVGFYQMAKIAVTPSIVFAEFLWYRKRVSFMKVVSLTVVSVGVAVATVTDLQFSLFGACVAFAWIIPSATNKILWSNMQQRENWTALALMWKTTPITLLFLVSMIPFLDPPGALSFNWSLTNTSAILVSALLGFFLQWSGALALGATSAITHVVLGQFKTCVLLLGNYYIFGSNSGFISVGGAFVAIMGTSLYTYLNTRGQSLKTSSSSSALSEKKSRFSDLKDDDKNLEPYGSEAV

SEQ ID NO: 13 - protein, 5 aa, Arabidopsis thaliana
  misc_feature (2)..(3): Xaa can be any naturally occurring amino acid
GXXKT

SEQ ID NO: 14 - protein, 5 aa, Arabidopsis thaliana
  misc_feature (2)..(2): Xaa can be His or Met
GXXKT

SEQ ID NO: 15 - protein, 5 aa, Arabidopsis thaliana
GHMKT

(19) United States
(12) Patent Application Publication
(10) Pub. No.: US 2015/0082494 A1
(43) Pub. Date: Mar. 19, 2015

(54) Cell Modified in the Expression of a Nucleotide Sugar Transporter

(71) Applicant: THE REGENTS OF THE UNIVERSITY OF CALIFORNIA, OAKLAND, CA (US)

(72) Inventors: Henrik Vibe Scheller, Millbrae, CA (US); Carsten Rautengarten, Oakland, CA (US); Berit Ebert, Oakland, CA (US); Joshua L. Heazlewood, Berkeley, CA (US)

(73) Assignee: THE REGENTS OF THE UNIVERSITY OF CALIFORNIA, OAKLAND, CA (US)

(21) Appl. No.: 14/306,183

(22) Filed: Jun. 16, 2014

Related U.S. Application Data
(60) Provisional application No. 61/835,506, filed on Jun. 14, 2013.

(51) Int. Cl. C12N 15/82

(52) U.S. Cl. CPC C12N 15/8245
USPC 800/306; 435/325; 435/254.11; 435/419; 800/298; 800/320; 800/320.2; 800/320.1; 800/314; 800/320.3; 800/317.3; 800/322; 435/455; 435/468; 435/471

(57) ABSTRACT

The present invention provides for a genetically modified eukaryotic host cell comprising (a) a gene encoding a first nucleotide sugar transporter (NST) operably linked to a promoter, wherein the gene and/or the promoter is heterologous to the cell, and/or (b) a native gene encoding a second NTS is disrupted and/or a promoter of the native gene is disrupted. Such modified cells can be altered in the production of polysaccharide and/or glycopeptides. The present invention also provides for methods of making or using such modified cells.

### RELATED PATENT APPLICATIONS

The application claims priority to U.S. Provisional Patent Application Ser. No. 61/835,506, filed Jun. 14, 2013, which is hereby incorporated by reference in its entirety.

### STATEMENT OF GOVERNMENTAL SUPPORT

The invention was made with government support under Contract No. DE-AC02-05CH11231 awarded by the U.S. Department of Energy. The government has certain rights in the invention.

### FIELD OF THE INVENTION

The present invention is in the field of cellular production of polysaccharide and/or glycopeptide.

### BACKGROUND OF THE INVENTION

Golgi nucleotide sugar transporter (NST) are important regulators of the cell wall biosynthesis for different reasons: (a) Non-cellulosic cell wall polymers/polysaccharides are synthesized in the Golgi as the enzymes (glycosyltransferases, GTs) that mediate the assembly of polysaccharides are located in the Golgi. (b) Activated sugars, the precursors for cell wall biosynthesis, however are mostly synthesized in the cytosol. Therefore transport into the Golgi lumen is essential for proper cell wall biosynthesis. (c) Targeted modulation of nucleotide sugar transport can help to modify cell wall biosynthesis and in combination with appropriate nucleotide sugar interconverting enzymes as well as glycosyltransferases lead to engineered plants which have altered contents of a targeted carbohydrate. (d) Knowing about the specific motifs for nucleotide sugar transport allows one to design highly active, mono-specific nucleotide sugar transporters and engineer plants with “regulated” nucleotide sugar transport. See FIG. 8.

Currently the plant cell wall can be modified by down-regulation or overexpression of enzymes (GTs) that mediate the assembly of polysaccharides. In addition, overexpression of Gals1, a beta-galactan synthase, leads to an increase in galactose in Arabidopsis cell walls. (U.S. Provisional Patent Application Ser. No. 61/645,537 and PCT International Patent Application No. PCT/US2013/40632; both titled “REGULATION OF GALACTAN SYNTHASE EXPRESSION TO MODIFY GALACTAN CONTENT IN PLANTS” and hereby incorporated by reference).

### SUMMARY OF THE INVENTION

The present invention provides for a genetically modified eukaryotic host cell comprising (a) a gene encoding a first nucleotide sugar transporter (NST) operably linked to a promoter, wherein the gene and/or the promoter is heterologous to the cell, and/or (b) a native gene encoding a second NTS is disrupted and/or a promoter of the native gene is disrupted. The modified cell is altered in producing a polysaccharide and/or glycolpeptide. The alteration is an increase or decrease in producing a polysaccharide and/or glycolpeptide. The polysaccharide or glycolpeptide can be a molecule that the unmodified cell produces or not produce. The polysaccharide or glycolpeptide can be naturally occurring or non-naturally occurring. When the modified cell is a plant cell, the polysaccharide can be a cell wall, or component thereof.

The present invention provides for a plant comprising the cell of the present invention, or a progeny thereof.

The present invention provides for a seed from the plant of the present invention.

The present invention provides for a biomass comprising plant tissue from the plant of the present invention.

The present invention provides for a method of obtaining a modified eukaryotic cell of the present invention, comprising: (a) introducing a nucleotide sugar transporter (NST) operably linked to a promoter into a eukaryotic cell, and (b) optionally culturing the eukaryotic cell.

In some embodiments, the method results in an increase of the amount of a polysaccharide or sugar in a plant comprising the plant cell.

In some embodiments, the method further comprises: (c) optionally growing the cultured plant cell into a plant, (d) optionally collecting the plant material from the plant, and (e) optionally incubating plant material from the plant in a saccharification reaction.

The present invention provides for a method of obtaining a modified eukaryotic host cell of the present invention, comprising: (a) disrupting a native gene encoding a nucleotide sugar transporter (NST) of a eukaryotic, or disrupting a promoter operably linked to the native gene, and (b) optionally culturing the eukaryotic cell.

The present invention provides for a method of altering the content of a polysaccharide or sugar in a plant, comprising: altering expression of an endogenous or heterologous nucleotide sugar transporter (NST) gene in the plant.

The present invention provides for a method of improving the amount of soluble sugar obtained from a plant biomass material, comprising: (a) providing plant biomass material from a plant in which an endogenous or heterologous nucleotide sugar transporter (NST) gene expression is increased, (b) performing a saccharification reaction, and (c) obtaining soluble sugar.

The present invention provides for a saccharification reaction comprising grass plant biomass material from a plant in which an endogenous or heterologous nucleotide sugar transporter (NST) gene expression is increased.

The present invention provides for a method of engineering a plant to increase the content of a sugar in a desired tissue, comprising: (a) introducing an expression cassette into the plant, wherein the expression cassette comprises a polynucleotide encoding a nucleotide sugar transporter (NST) operably linked to a heterologous promoter, wherein the NST has at least 70% identity to a sequence of a naturally occurring NST amino acid sequence, and culturing the plant under conditions in which the NST is expressed.

The present invention provides for a plant cell comprising: (a) a polynucleotide encoding a NST operably linked to a heterologous promoter, or (b) a heterologous polynucleotide encoding a NST protein.

The present invention provides for a method of obtaining an increased amount of soluble sugars from a plant in a saccharification reaction, comprising: subjecting the plant of one of claims 4-6 to a saccharification reaction, thereby increasing the amount of soluble sugars that can be obtained from the plant as compared to a wild-type plant

This present invention provides for engineering cell and/or plants by specifically modifying nucleotide sugar transport into the Golgi lumen. The invention provides for a means to target more than one GT activity and different polymers at the same time as it allows regulation at an earlier step than the assembly of the cell wall polymers, and optionally modulate the availability of a certain substrate, such as by varying transport rates.

The present invention provides a method that allows the modifying of a plant cell wall by specifically changing nucleotide sugar transport into the Golgi lumen for certain nucleotide sugars. The method is based on the specificity of the NST, which in turn can be used to increase or decrease a certain sugar in cell wall polymers that are synthesized in the Golgi. Co-expression of glycosyltransferases and the appropriate NST overcomes possible limitations caused by restricted availabilities of nucleotide sugar substrates within the Golgi lumen.

The present invention provides a novel way of achieving plant cell wall modification. Nucleotide sugar transporters are employed for cell wall engineering so far as transport activities had been shown for only a few nucleotide sugars and their effects on the plant cell wall has not been studied. Novel NSTs are identified and demonstrated that they are capable of transporting UDP-Rhamnose, UDP-Xylose, and UDP-Arabinose. In addition, plants are analyzed, which lack the functional protein for a respective gene encoding for a NST as well as plants overexpressing the protein.

Knowledge about NSTs provides guidance on designing plants with more desirable characteristics for biofuel production as it allows the specific modification of nucleotide sugar transport. It is shown that for GalT2 an UDP-Galactose/UDP Rhamnose transporter, plants overexpressing this NST can accumulate up to 50% more galactose in leaves. This level of accumulation is higher than what could be achieved by overexpressing GALS 1 (a galactan synthase). Hence, overexpression of both the transport and the synthase (and optionally also UDP-glucose epimerase at the same time) is predicted to give even better results). The overexpression of galactan synthase is taught in U.S. Provisional Patent Application Ser. No. 61/645,537 and PCT International Patent Application No. PCT/US2013/40632; both titled “REGULATION OF GALACTAN SYNTHASE EXPRESSION TO MODIFY GALACTAN CONTENT IN PLANTS” and hereby incorporated by reference.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1 shows the monosaccharide composition obtained using the galT2-1 and galT2-2. Galactose is significantly reduced in the galT2-1 and galT2-2 mutants.

FIG. 2 shows that overexpression of GalT2 results in increased galactose content. Plants overexpressing GalT2 accumulate up to 50% more galactose in leaves compared to plants not overexpressing GalT2.

FIG. 3 shows GalT2 affects specific polymers.

FIG. 4 shows the results of galactanase treatment. The results indicate that GalT2 preferentially affects β-1,4-galactan biosynthesis in vivo.

FIG. 5 shows the results of OLIMP analysis.

FIG. 6 shows the predicted phylogenetic relationship tree of Arabidopsis NSTs. 14 NSTs (indicated by the arrows) are identified in the Golgi proteome. NST belonging to the NST-KY subfamily of clade are circled.

FIG. 7 shows the multiple alignments of the amino acid sequences of a set of NST of the NST-KT subfamily or clade. The conserved lysine/threonine (KT) motif is boxed.

FIG. 8 shows the model of NST function in the Golgi apparatus. The transport of NDP sugars from the cytosol into the lumen of the Golgi apparatus is essential for proper cell wall biosynthesis.

### DETAILED DESCRIPTION OF THE INVENTION

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

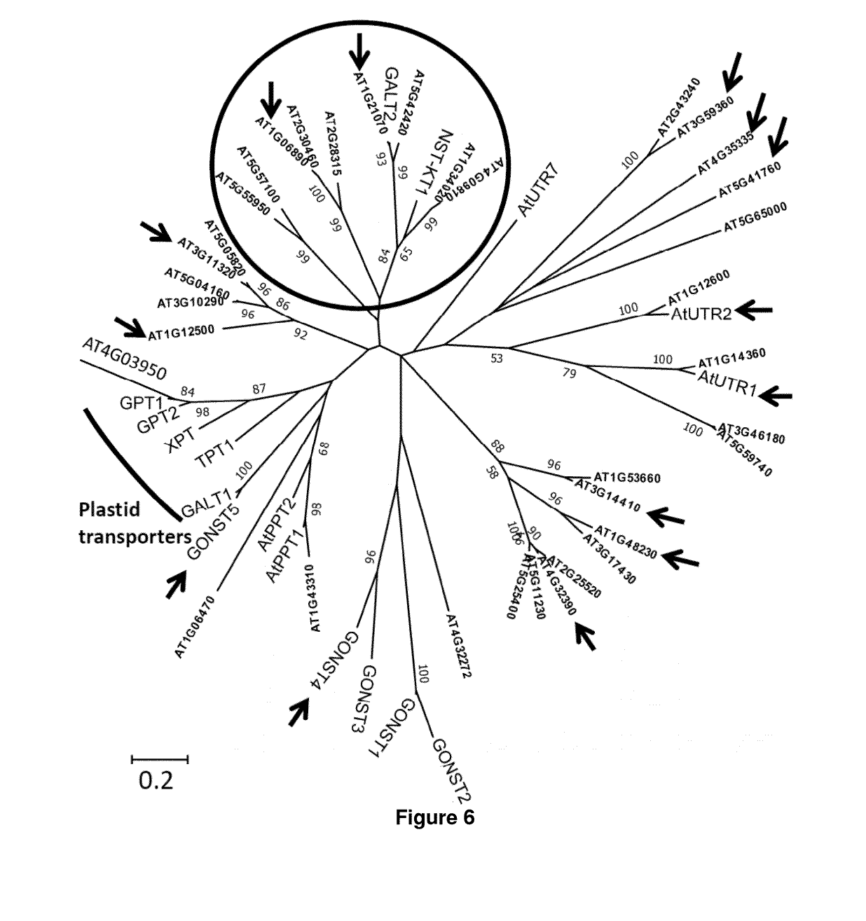

As used herein, the term “nucleotide sugar transporter” or “NST” are used interchangeably to refer to an enzyme that is involved in the transport of a NDP sugar from the cytosol into the Golgi body. The term encompasses polymorphic variants, alleles, mutants, and interspecies homologs to the specific polypeptides described herein. A nucleic acid that encodes a NST refers to a gene, pre-mRNA, mRNA, and the like, including nucleic acids encoding polymorphic variants, alleles, mutants, and interspecies homologs of the particular amino acid sequences described herein. Thus, in some embodiments, a NST encodes a polypeptide having an amino acid sequence that has at least 50% amino acid sequence identity, or at least 55%, 60%, 65%, 70%, 75%, 80%, 85%, 90%, preferably 91%, 92%, 93%, 94%, 95%, 96%, 97%, 98% or 99% or greater amino acid sequence identity, preferably over a region of at least about 25, 50, 100, 200 or more amino acids, or over the length of the entire polypeptide, to any one of the amino acid sequences of the NSTs shown in Table 1 and FIG. 6.

The terms “increased level of activity,” or “increased activity” refer interchangeably to an increase in the amount of activity of NST protein in a cell or plant engineered to increase transport of one or more NDP sugar compared to the amount of activity in a wild-type (i.e., naturally occurring) cell or plant. In some embodiments, increased activity results from increased expression levels. An increased level of activity or increased level of expression can be an increase in the amount of activity or expression of NST in a cell or plant genetically modified to overexpress NST of at least 10%, 20%, 30%, 40%, 50%, 60%, 70%, 80%, or 90% or greater compared to a wildtype cell or plant. In some embodiments, the increased NST activity or expression is localized to one or more tissues of the engineered plant, such as the xylem cells with secondary cell walls. Increased expression or activity of a NST gene or protein can be assessed by any number of assays, including, but not limited to, the methods described in Examples 1 and 2.

The terms “reduced level of activity,” “reduced activity” and “decreased activity” refer interchangeably to a reduction in the amount of activity of NST protein in a cell or plant engineered to decrease NST compared to the amount of activity in a wild-type (i.e., naturally occurring) cell or plant. In some embodiments, reduced activity results from reduced expression levels. A reduced level of activity or a reduced level of expression can be a reduction in the amount of activity or expression of NST of at least 10%, 20%, 30%, 40%, 50%, 60%, 70%, 80%, or 90% or greater. In some embodiments, the reduced level of activity or reduced level of expression occurs, throughout all the tissues of the engineered cell or plant. In some embodiments, the reduction in the amount of activity or expression is localized to one or more tissues of the engineered plant, such as the cell wall. In some embodiments, the NST is not reduced in amount, but is modified in amino acid sequence so that its activity is reduced directly or indirectly. Decreased expression or activity of a NST gene or protein can be assessed by any number of assays, including, but not limited to, the methods described in Examples 1 and 2.

The terms “polynucleotide” and “nucleic acid” are used interchangeably and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); positive backbones; non-ionic backbones, and non-ribose backbones. Thus, nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc.

The term “substantially identical,” used in the context of two nucleic acids or polypeptides, refers to a sequence that has at least 50% sequence identity with a reference sequence. Percent identity can be any integer from 50% to 100%. Some embodiments include at least: 50%, 55%, 60%, 65%, 70%, 75%, 80%, 85%, 90%, 91%, 92%, 93%, 94%, 95%, 96%, 97%, 98%, or 99%, compared to a reference sequence using the programs described herein; preferably BLAST using standard parameters, as described below. For example, a polynucleotide encoding a NST polypepitde may have a sequence that is at least 50%, 55%, 60%, 65%, 70%, 75%, 80%, 85%, 90%, 91%, 92%, 93%, 94%, 95%, 96%, 97%, 98%, or 99% identical to a sequence of a NST identified in Table 1 and FIG. 6.

Two nucleic acid sequences or polypeptide sequences are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, Calif., USA).

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

Nucleic acid or protein sequences that are substantially identical to a reference sequence include “conservatively modified variants.” With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, one of skill will recognize that individual substitutions, in a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are illustrative conservative substitutions for one another: (1) Alanine (A), Serine (S), Threonine (T); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); and (6) Phenylalanine (F), Tyrosine (Y), Tryptophan (W). (see, e.g., Creighton, Proteins (1984)).

Another indication that nucleotide sequences are substantially identical is if two molecules hybridize to each other, or a third nucleic acid, under stringent conditions. Stringent conditions are sequence dependent and will be different in different circumstances. Generally, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent conditions will be those in which the salt concentration is about 0.02 molar at pH 7 and the temperature is at least about 60° C. For example, stringent conditions for hybridization, such as RNA-DNA hybridizations in a blotting technique are those which include at least one wash in 0.2×SSC at 55° C. for 20 minutes, or equivalent conditions.

The term “promoter,” as used herein, refers to a polynucleotide sequence capable of driving transcription of a DNA sequence in a cell. Thus, promoters used in the polynucleotide constructs of the invention include cis- and trans-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. Promoters are located 5′ to the transcribed gene, and as used herein, include the sequence 5′ from the translation start codon (i.e., including the 5′ untranslated region of the mRNA, typically comprising 100-200 bp). Most often the core promoter sequences lie within 1-2 kb of the translation start site, more often within 1 kbp and often within 500 bp of the translation start site. By convention, the promoter sequence is usually provided as the sequence on the coding strand of the gene it controls. In the context of this application, a promoter is typically referred to by the name of the gene for which it naturally regulates expression. A promoter used in an expression construct of the invention is referred to by the name of the gene. Reference to a promoter by name includes a wildtype, native promoter as well as variants of the promoter that retain the ability to induce expression. Reference to a promoter by name is not restricted to a particular species, but also encompasses a promoter from a corresponding gene in other species.

A “constitutive promoter” in the context of this invention refers to a promoter that is capable of initiating transcription in nearly all cell types, whereas a “cell type-specific promoter” or “tissue-specific promoter” initiates transcription only in one or a few particular cell types or groups of cells forming a tissue (for a multi-cellular organism). In some embodiments, a plant promoter is tissue-specific if the transcription levels initiated by the promoter in the cell wall are at least 2-fold, 3-fold, 4-fold, 5-fold, 6-fold, 7-fold, 8-fold, 9-fold, 10-fold, 50-fold, 100-fold, 500-fold, 1000-fold higher or more as compared to the transcription levels initiated by the promoter in non-cell wall tissues

A polynucleotide is “heterologous” to an organism or a second polynucleotide sequence if it originates from a foreign species, or, if from the same species, is modified from its original form. For example, when a polynucleotide encoding a polypeptide sequence is said to be operably linked to a heterologous promoter, it means that the polynucleotide coding sequence encoding the polypeptide is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety).

The term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a DNA or RNA sequence if it stimulates or modulates the transcription of the DNA or RNA sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The term “expression cassette” or “DNA construct” or “expression construct” refers to a nucleic acid construct that, when introduced into a host cell, results in transcription and/or translation of an RNA or polypeptide, respectively. Antisense or sense constructs that are not or cannot be translated are expressly included by this definition. In the case of both expression of transgenes and suppression of endogenous genes (e.g., by antisense, RNAi, or sense suppression) one of skill will recognize that the inserted polynucleotide sequence need not be identical, but may be only substantially identical to a sequence of the gene from which it was derived. As explained herein, these substantially identical variants are specifically covered by reference to a specific nucleic acid sequence. One example of an expression cassette is a polynucleotide construct that comprises a polynucleotide sequence encoding a NST protein operably linked to a heterologous promoter. In some embodiments, an expression cassette comprises a polynucleotide sequence encoding a NST protein that is targeted to a position in a plant genome such that expression of the polynucleotide sequence is driven by a promoter that is present in the plant

The term “plant” as used herein can refer to a whole plant or part of a plant, e.g., seeds, and includes plants of a variety of ploidy levels, including aneuploid, polyploid, diploid and haploid. The term “plant part,” as used herein, refers to shoot vegetative organs and/or structures (e.g., leaves, stems and tubers), branches, roots, flowers and floral organs (e.g., bracts, sepals, petals, stamens, carpels, anthers), ovules (including egg and central cells), seed (including zygote, embryo, endosperm, and seed coat), fruit (e.g., the mature ovary), seedlings, and plant tissue (e.g., vascular tissue, ground tissue, and the like), as well as individual plant cells, groups of plant cells (e.g., cultured plant cells), protoplasts, plant extracts, and seeds. The class of plants that can be used in the methods of the invention is generally as broad as the class of higher and lower plants amenable to transformation techniques, including angiosperms (monocotyledonous and dicotyledonous plants), gymnosperms, ferns, bryophytes, and multicellular algae.

The term “biomass,” as used herein, refers to plant material that is processed to provide a product, e.g., a biofuel such as ethanol, or livestock feed, or a cellulose for paper and pulp industry products. Such plant material can include whole plants, or parts of plants, e.g., stems, leaves, branches, shoots, roots, tubers, and the like.

The term “increased cell wall deposition” in the context of galactan deposition refers to an increased amount of galactan in a cell wall that is produced in an engineered plant of the present invention as compared to a wild-type (i.e., naturally occurring) plant. In the current invention, galactan deposition is typically considered to be increased when the amount of galactan in the cell wall is increased by at least 10%, at least 20, 30%, 40%, 50%, 60%, 70%, 80%, 90% or more relative to the amount of galactan in the cell wall in a wild-type plant. The amount of galactan can be assessed using any method known in the art, including using an antibody that specifically binds galactan or enzymatic or chemical analyses.

The term “saccharification reaction” refers to a process of converting biomass, usually cellulosic or lignocellulosic biomass, into monomeric sugars, such as glucose and xylose.

The term “soluble sugar” refers to monomeric, dimeric, or trimeric sugar that is produced from the saccharification of biomass.

The term “increased amount,” when referring to an amount of sugar or soluble sugar obtained from an engineered plant of the present invention, refers to an increase in the amount or yield of sugar that is obtained from saccharification of biomass per amount of starting material, in comparison to corresponding biomass from a wild-type (i.e., naturally occurring) plant. In the context of the present invention, “corresponding biomass from a wild-type plant” refers to plant material that is from the same part of the plant as the biomass from a plant engineered to have modified sugar levels. As understood in the art, increased amount or increased yield is based upon comparisons of the same amount of corresponding plant material.

The term “conversion reaction,” as used herein, refers to a reaction that converts biomass into a form of bioenergy. Examples of conversion reactions include, but are not limited to, combustion (burning), gasification, pyrolysis, and polysaccharide hydrolysis (enzymatic or chemical).

The term “increased production,” when referring to an amount of bioenergy production obtained from an engineered plant of the present invention, refers to an increased amount of bioenergy that is produced from subjecting biomass from an engineered plant to a conversion reaction (e.g., combustion, gasification, pyrolysis, or polysaccharide hydrolysis) as compared to the amount of bioenergy that is produced from corresponding biomass from a wild-type (i.e., naturally occurring) plant.

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

These and other objects, advantages, and features of the invention will become apparent to those persons skilled in the art upon reading the details of the invention as more fully described below.

### Nucleotide Sugar Transporters (NST)

Identification of genes encoding NSTs allows the specific modification of transport for certain nucleotide sugars. It is beneficial to have methods to engineer plants with a desirable amount of a certain sugar or restrict a certain sugar only to specific polymers or tissues. Knowledge about what regulates the specificity of a NST will be helpful and also specific expression of a NST under a tissue specific promoter could be helpful. Also the design of monospecific NSTs could be a solution to overcome these issues.

This invention is useful for engineering bioenergy plants with a cell wall composition that makes their sugars easier accessible. This will be interesting for a variety of industries, such as biofuel production or sugar producing industries. Likewise, the invention could be useful for developing plants for other purposes, such as for feed and forage.

The invention can also be useful in providing parts for synthetic biology in other organisms. Thus, for example UDP-GalA and UDP-Rha transporters are not present in yeast and generation of glycosylated proteins or polysaccharides with GalA or Rha in such an organism can be achieved using the transporters and methods described herein.

In an aspect of the invention is also be useful in providing “clean” glycosylated protein or peptide in a non-mammalian or non-animal host cell, such as a yeast or plant host cell. The “clean” glycosylated protein or peptide is a protein or peptide that is not glycoslylated by mammalian and/or animal unique glycosylated transferase.

The present invention also provides for a glycosylated protein produced by a host cell and/or method of the present invention.

In one aspect, the invention provides a method of engineering plants to increase galactan content, e.g., to improve biofuel potential. Eukaryotic cells can be engineered to overexpress or reduce express one or more NST in a cell by genetically modifying the cell to overexpress or disrupt one or more NST genes as described herein. In some embodiments, plants can be engineered to overexpress or reduce express one or more NST in the plant by genetically modifying the plant to overexpress or disrupt one or more NST genes as described herein. Typically, overexpression is targeted to cell wall using a tissue-specific promoter. An example of a method for fine-tuning gene expression to increase expression in the cell wall is taught in PCT/US2012/023182, which is incorporated by reference.

A plant that is engineered to overexpress a NST and a sugar synthase, such as GALS, may also be engineered to overexpress a UDP-galactose epimerase (more commonly referred to as a UDP-glucose epimerase). Such epimerases are well known in the art. Examples of epimerase genes are described by Barber et al., J. Biol. Chem. 281:17276-17285, 2006 and Kotake et al., Biochem. J. 424:169-177, 2009, each of which is incorporated by reference.

In a further aspect, a plant may be further modified to alter the enzymes that synthesize nucleotide sugar substrates. Such enzymes could include UDP-glucose pyrophosphorylase and other non-specific UDP-sugar pyrophosphorylases.

In some embodiments, a cell is modified to express a heterologous NST so that the modified cell can transport a nucleotide sugar that does not naturally occur in the wild-type cell in order to produce a polysaccharide or glycopeptide that is not naturally occurring. In some embodiments, the modified cell, or a library of such modified cells, can be modified to produce a library of polysaccharides and/or glycopeptides. Such polysaccharides and/or glycopeptides, or libraries thereof, can be further screened for any particularly activity, such as binding to or compete with to an epitope or molecule of interest. One example of such a screen is a screen for a suitable anti-cancer drug.

### NST Nucleic Acid Sequences

The invention employs various routine recombinant nucleic acid techniques. Generally, the nomenclature and the laboratory procedures in recombinant DNA technology described below are those well known and commonly employed in the art. Many manuals that provide direction for performing recombinant DNA manipulations are available, e.g., Sambrook & Russell, Molecular Cloning, A Laboratory Manual (3rd Ed, 2001); and Current Protocols in Molecular Biology (Ausubel, et al., John Wiley and Sons, New York, 2009).

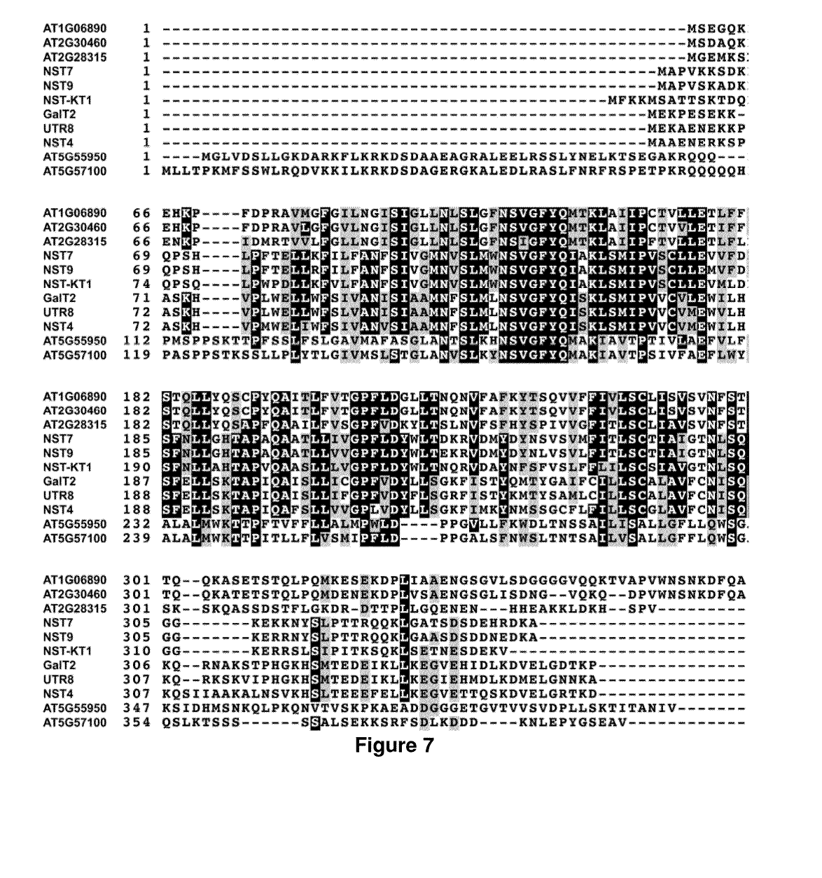

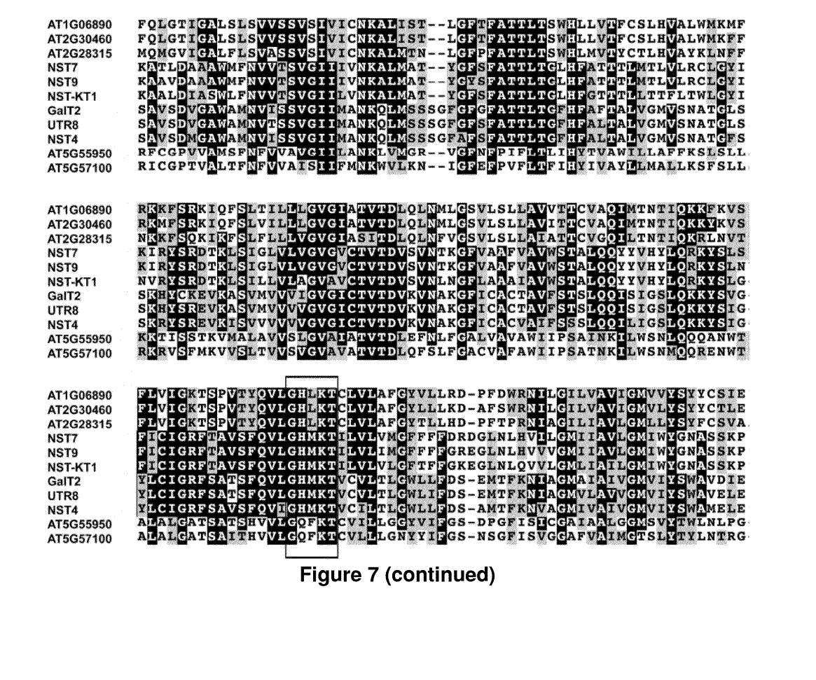

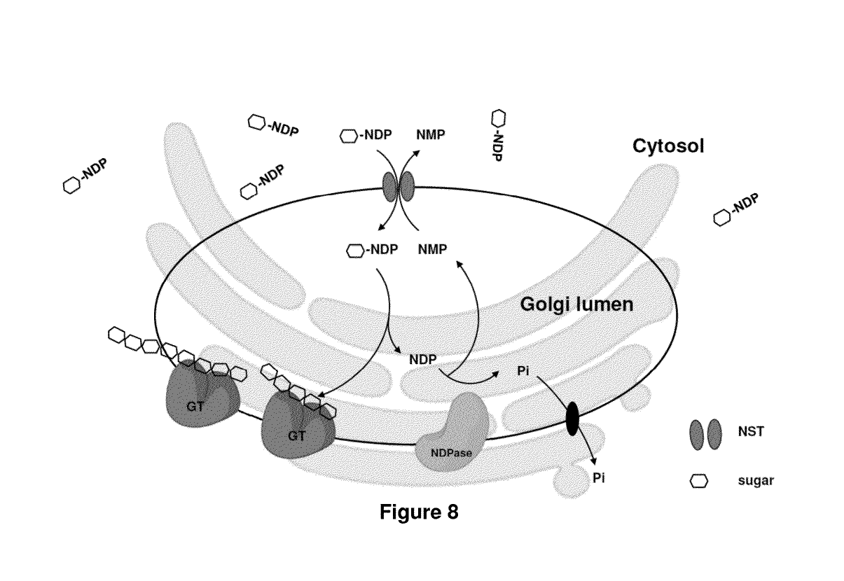

In some embodiments, the first NST is a plant, animal or fungal NST. In some embodiments, the NST is a NST of Arabidopsis, poplar, eucalyptus, rice, corn, cotton, switchgrass, sorghum, millet, miscanthus, sugarcane, pine, alfalfa, wheat, soy, barley, turfgrass, tobacco, hemp, bamboo, rape, sunflower, willow, or Brachypodium. In some embodiments, the NST is of the NST-lysine-threonine (KT) subfamily or clade. The amino acid sequence of certain members of the NST-KT subfamily or clade are shown in FIG. 7. NST of the NST-KT subfamily or clade comprise the KT motif, which comprises the amino acid sequence GXXKT or G(H or M)(L, M or F)KT. In some embodiments, the NST comprises the amino acid sequence GHMKT.

NST nucleic acid and polypeptide sequences suitable for use in the invention include NST nucleic acid sequences that encode a plant, animal or yeast NST polypeptide, or a substantially identical variant thereof. Such a variant typically has at least 60%, often at least 70%, or at least 75%, 80%, 85%, or 90% identity to the amino acid sequences of any one of the NST identified in Table 1 or FIG. 7. The amino acid sequence of any of the NST identified in Table 1 can be easily obtained by using the AGI code of the corresponding NST to obtain its amino acid sequence from The Arabidopsis Information Resource (TAR) website (website for arabidopsis.org). For example, the amino acid sequence of GalT2 (SEQ ID NO:1) is:

```
    1 MEKPESEKKS AVSDVGAWAM NVISSVGIIM ANKQLMSSSG
FGFGFATTLT
  51 GFHFAFTALV GMVSNATGLS ASKHVPLWEL LWFSIVANIS
IAAMNFSLML
101 NSVGFYQISK LSMIPVVCVL EWILHSKHYC KEVKASVMVV
VIGVGICTVT
151 DVKVNAKGFI CACTAVFSTS LQQISIGSLQ KKYSVGSFEL
LSKTAPIQAI
201 SLLICGPFVD YLLSGKFIST YQMTYGAIFC ILLSCALAVF
CNISQYLCIG
251 RFSATSFQVL GHMKTVCVLT LGWLLFDSEM TFKNIAGMAI
AIVGMVIYSW
301 AVDIEKQRNA KSTPHGKHSM TEDEIKLLKE GVEHIDLKDV
ELGDTKP
```

NSTs can include proteins that transport any nucleotide sugar, including, but not limited to UDP-Glc, UDP-Gal, UDP-GlcA, UDP-GalA, UDP-Rha, UDP-Arap, UDP-Araf, GDP-Man, GDP-Fuc, UDP-GlcNAc, GDP-Glc, UDP-Api, ADP-Glc, GDP-Gal, UDP-GalNAc, CMP-Kdo, CMP-DHA, CMP-Sialic acid, and the like. The nucleotide sugar can be naturally occurring or non-naturally occurring. A heterologous NST can be introduced into a cell so that the modified cell expresses the heterologous NST.

NST activity can be assessed using any number of assays, including assays described in Example 1. Isolation or generation NST polynucleotide sequences can be accomplished by a number of techniques. Cloning and expression of such technique is addressed in the context of NST genes. In some embodiments, oligonucleotide probes based on the sequences disclosed here can be used to identify the desired polynucleotide in a cDNA or genomic DNA library from a desired plant species. Probes may be used to hybridize with genomic DNA or cDNA sequences to isolate homologous genes in the same or different plant species.

Alternatively, the nucleic acids of interest can be amplified from nucleic acid samples using routine amplification techniques. For instance, PCR may be used to amplify the sequences of the genes directly from mRNA, from cDNA, from genomic libraries or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes.

Appropriate primers and probes for identifying a NST gene from plant cells can be generated from comparisons of the sequences provided herein. For a general overview of PCR see PCR Protocols: A Guide to Methods and Applications. (Innis, M, Gelfand, D., Sninsky, J. and White, T., eds.), Academic Press, San Diego (1990).

NST nucleic acid sequences for use in the invention includes genes and gene products identified and characterized by techniques such as hybridization and/or sequence analysis using exemplary nucleic acid sequences taught herein.

### Preparation of Recombinant Vectors

To use isolated sequences in the above techniques, recombinant DNA vectors suitable for transformation of plant cells such as crop plant cells are prepared. Techniques for transformation are well known and described in the technical and scientific literature. For example, a DNA sequence encoding a NST polypepitde can be combined with transcriptional and other regulatory sequences which will direct the transcription of the sequence from the gene in the intended cells, e.g., grass or other crop plant cells. In some embodiments, an expression vector that comprises an expression cassette that comprises the NST gene further comprises a promoter operably linked to the NST gene. In other embodiments, a promoter and/or other regulatory elements that direct transcription of the NST gene are endogenous to the plant and an expression cassette comprising the NST gene is introduced, e.g., by homologous recombination, such that the heterologous NST gene is operably linked to an endogenous promoter and is expression driven by the endogenous promoter. Regulatory sequences include promoters, which may be either constitutive or inducible, or tissue-specific.

### Tissue-Specific Promoters

In some embodiments, a plant promoter to direct expression of a NST gene in a specific tissue is employed (tissue-specific promoters). Tissue specific promoters are transcriptional control elements that are only active in particular cells or tissues at specific times during plant development, such as in vegetative tissues or reproductive tissues.

Examples of tissue-specific promoters under developmental control include promoters that initiate transcription only (or primarily only) in certain tissues, such as vegetative tissues, cell walls, including e.g., roots or leaves. A variety of promoters specifically active in vegetative tissues, such as leaves, stems, roots and tubers are known. For example, promoters controlling patatin, the major storage protein of the potato tuber, can be used (see, e.g., Kim, Plant Mol. Biol. 26:603-615, 1994; Martin, Plant J. 11:53-62, 1997). The ORF13 promoter from Agrobacterium rhizogenes that exhibits high activity in roots can also be used (Hansen, Mol. Gen. Genet. 254:337-343, 1997). Other useful vegetative tissue-specific promoters include: the tarin promoter of the gene encoding a globulin from a major taro (Colocasia esculenta L. Schott) corm protein family, tarin (Bezerra, Plant Mol. Biol. 28:137-144, 1995); the curculin promoter active during taro corm development (de Castro, Plant Cell 4:1549-1559, 1992) and the promoter for the tobacco root-specific gene TobRB7, whose expression is localized to root meristem and immature central cylinder regions (Yamamoto, Plant Cell 3:371-382, 1991).

Leaf-specific promoters, such as the ribulose biphosphate carboxylase (RBCS) promoters can be used. For example, the tomato RBCS1, RBCS2 and RBCS3A genes are expressed in leaves and light-grown seedlings, only RBCS1 and RBCS2 are expressed in developing tomato fruits (Meier, FEBS Lett. 415:91-95, 1997). A ribulose bisphosphate carboxylase promoters expressed almost exclusively in mesophyll cells in leaf blades and leaf sheaths at high levels (e.g., Matsuoka, Plant J. 6:311-319, 1994), can be used. Another leaf-specific promoter is the light harvesting chlorophyll a/b binding protein gene promoter (see, e.g., Shiina, Plant Physiol. 115:477-483, 1997; Casal, Plant Physiol. 116:1533-1538, 1998). The Arabidopsis thaliana myb-related gene promoter (Atmyb5) (Li, et al., FEBS Lett. 379:117-121 1996), is leaf-specific. The Atmyb5 promoter is expressed in developing leaf trichomes, stipules, and epidermal cells on the margins of young rosette and cauline leaves, and in immature seeds. Atmyb5 mRNA appears between fertilization and the 16 cell stage of embryo development and persists beyond the heart stage. A leaf promoter identified in maize (e.g., Busk et al., Plant J. 11:1285-1295, 1997) can also be used.

Another class of useful vegetative tissue-specific promoters are meristematic (root tip and shoot apex) promoters. For example, the “SHOOTMERISTEMLESS” and “SCARECROW” promoters, which are active in the developing shoot or root apical meristems, (e.g., Di Laurenzio, et al., Cell 86:423-433, 1996; and, Long, et al., Nature 379:66-69, 1996); can be used. Another useful promoter is that which controls the expression of 3-hydroxy-3-methylglutaryl coenzyme A reductase HMG2 gene, whose expression is restricted to meristematic and floral (secretory zone of the stigma, mature pollen grains, gynoecium vascular tissue, and fertilized ovules) tissues (see, e.g., Enjuto, Plant Cell. 7:517-527, 1995). Also useful are kn1-related genes from maize and other species which show meristem-specific expression, (see, e.g., Granger, Plant Mol. Biol. 31:373-378, 1996; Kerstetter, Plant Cell 6:1877-1887, 1994; Hake, Philos. Trans. R. Soc. Lond. B. Biol. Sci. 350:45-51, 1995). For example, the Arabidopsis thaliana KNAT1 promoter (see, e.g., Lincoln, Plant Cell 6:1859-1876, 1994) can be used.

In some embodiments, the promoter is substantially identical to the native promoter of a promoter that drives expression of a gene involved in secondary wall deposition. Examples of such promoters are promoters from IRX1, IRX3, IRX5, IRX8, IRX9, IRX14, IRX7, IRX10, GAUT13, or GAUT14 genes. Specific expression in fiber cells can be accomplished by using a promoter such as the NST1 promoter and specific expression in vessels can be accomplished by using a promoter such as VND6 or VND7. (See, e.g., PCT/US2012/023182 for illustrative promoter sequences).

One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

### Constitutive Promoters

A promoter, or an active fragment thereof, can be employed which will direct expression of a nucleic acid encoding a fusion protein of the invention, in all or most transformed cells or tissues, e.g. as those of a regenerated plant. Such promoters are referred to herein as “constitutive” promoters and are active under most environmental conditions and states of development or cell differentiation. Examples of constitutive promoters include those from viruses which infect plants, such as the cauliflower mosaic virus (CaMV) 35S transcription initiation region (see, e.g., Dagless, Arch. Virol. 142:183-191, 1997); the 1′- or 2′-promoter derived from T-DNA of Agrobacterium tumefaciens (see, e.g., Mengiste supra (1997); O'Grady, Plant Mol. Biol. 29:99-108, 1995); the promoter of the tobacco mosaic virus; the promoter of Figwort mosaic virus (see, e.g., Maiti, Transgenic Res. 6:143-156, 1997); actin promoters, such as the Arabidopsis actin gene promoter (see, e.g., Huang, Plant Mol. Biol. 33:125-139, 1997); alcohol dehydrogenase (Adh) gene promoters (see, e.g., Millar, Plant Mol. Biol. 31:897-904, 1996); ACT11 from Arabidopsis (Huang et al., Plant Mol. Biol. 33:125-139, 1996), Cat3 from Arabidopsis (GenBank No. U43147, Zhong et al., Mol. Gen. Genet. 251:196-203, 1996), the gene encoding stearoyl-acyl carrier protein desaturase from Brassica napus (Genbank No. X74782, Solocombe et al., Plant Physiol. 104:1167-1176, 1994), GPc1 from maize (GenBank No. X15596, Martinez et al., J. Mol. Biol. 208:551-565, 1989), Gpc2 from maize (GenBank No. U45855, Manjunath et al., Plant Mol. Biol. 33:97-112, 1997), other transcription initiation regions from various plant genes known to those of skill See also Holtorf, “Comparison of different constitutive and inducible promoters for the overexpression of transgenes in Arabidopsis thaliana,” Plant Mol. Biol. 29:637-646, 1995).

### Inducible Promoters

In some embodiments, a plant promoter may direct expression of the nucleic acids under the influence of changing environmental conditions or developmental conditions. Examples of environmental conditions that may effect transcription by inducible promoters include anaerobic conditions, elevated temperature, drought or other environmental stress, or the presence of light. Examples of developmental conditions that may effect transcription by inducible promoters include senescence and embryogenesis. Such promoters are referred to herein as “inducible” promoters. For example, the invention can incorporate drought-specific promoter such as the drought-inducible promoter of maize (Busk et al., Plant J, 11: 1285-95, 1997); or alternatively the cold, drought, and high salt inducible promoter from potato (Kirch Plant Mol. Biol. 33:897-909, 1997).

Suitable promoters responding to biotic or abiotic stress conditions include the pathogen inducible PRP1-gene promoter (Ward et al., Plant. Mol. Biol. 22:361-366, 1993), the heat inducible hsp80-promoter from tomato (U.S. Pat. No. 5,187,267), cold inducible alpha-amylase promoter from potato (PCT Publication No. WO 96/12814) or the wound-inducible pinII-promoter (European Patent No. 375091). For other examples of drought, cold, and salt-inducible promoters, such as the RD29A promoter, see, e.g., Yamaguchi-Shinozalei et al., Mol. Gen. Genet. 236:331-340, 1993 are also known.

Alternatively, plant promoters which are inducible upon exposure to plant hormones, such as auxins, may be used to express NST genes. For example, the invention can use the auxin-response elements E1 promoter fragment (AuxREs) in the soybean (Glycine max L.) (Liu, Plant Physiol. 115:397-407, 1997); the auxin-responsive Arabidopsis GST6 promoter (also responsive to salicylic acid and hydrogen peroxide) (Chen, Plant J. 10: 955-966, 1996); the auxin-inducible parC promoter from tobacco (Sakai, 37:906-913, 1996); a plant biotin response element (Streit, Mol. Plant Microbe Interact. 10:933-937, 1997); and, the promoter responsive to the stress hormone abscisic acid (Sheen, Science 274:1900-1902, 1996).

Plant promoters inducible upon exposure to chemicals reagents that may be applied to the plant, such as herbicides or antibiotics, are also useful for expressing a NST gene in accordance with the invention. For example, the maize In2-2 promoter, activated by benzenesulfonamide herbicide safeners, can be used (De Veylder, Plant Cell Physiol. 38:568-577, 19997); application of different herbicide safeners induces distinct gene expression patterns, including expression in the root, hydathodes, and the shoot apical meristem. A NST coding sequence can also be under the control of, e.g., a tetracycline-inducible promoter, such as described with transgenic tobacco plants containing the Avena sativa L. (oat) arginine decarboxylase gene (Masgrau, Plant J. 11:465-473, 1997); or, a salicylic acid-responsive element (Stange, Plant J. 11:1315-1324, 1997; Uknes et al., Plant Cell 5:159-169, 1993); Bi et al., Plant J. 8:235-245, 1995).

Examples of useful inducible regulatory elements include copper-inducible regulatory elements (Mett et al., Proc. Natl. Acad. Sci. USA 90:4567-4571, 1993); Furst et al., Cell 55:705-717, 1988); tetracycline and chlor-tetracycline-inducible regulatory elements (Gatz et al., Plant J. 2:397-404, 1992); Wider et al., Mol. Gen. Genet. 243:32-38, 1994); Gatz, Meth. Cell Biol. 50:411-424, 1995); ecdysone inducible regulatory elements (Christopherson et al., Proc. Natl. Acad. Sci. USA 89:6314-6318, 1992; Kreutzweiser et al., Ecotoxicol. Environ. Safety 28:14-24, 1994); heat shock inducible regulatory elements (Takahashi et al., Plant Physiol. 99:383-390, 1992; Yabe et al., Plant Cell Physiol. 35:1207-1219, 1994; Ueda et al., Mol. Gen. Genet. 250:533-539, 1996); and lac operon elements, which are used in combination with a constitutively expressed lac repressor to confer, for example, IPTG-inducible expression (Wilde et al., EMBO J. 11:1251-1259, 1992). An inducible regulatory element useful in the transgenic plants of the invention also can be, for example, a nitrate-inducible promoter derived from the spinach nitrite reductase gene (Back et al., Plant Mol. Biol. 17:9 (1991)) or a light-inducible promoter, such as that associated with the small subunit of RuBP carboxylase or the LHCP gene families (Feinbaum et al., Mol. Gen. Genet. 226:449 (1991); Lam and Chua, Science 248:471 (1990)).

### Expression Using a Positive Feedback Loop

In further embodiments, a plant can be engineered to overexpress NST using a positive feedback loop to express NST in a desired tissue. In some embodiments, a promoter for use in a NST expression construct is responsive to a transcription factor that mediates expression in the desired tissue. The NST expression construct is used in a genetically modified plant comprising an expression construct encoding a transcription factor where expression is also driven by a promoter that is responsive to the transcription factor. Examples of such expression systems are provided in PCT/US2012/023182, hereby incorporated by reference.

In some embodiments in which a positive feedback loop is employed, the plant is genetically modified to express a transcription factor that regulates the production of secondary cell wall. Examples of such transcription factors include NST1, NST2, NST3, SND2, SND3, MYB103, MBY85, MYB46, MYB83, MYB58, and MYB63 (See, e.g., Mitsuda et al., Plant Cell 17:2993-3006 (2005); Mitsuda et al., Plant Cell 19:270-80 (2007); Ohashi-Ito et al., Plant Cell 22:3461-73 (2010); Zhong et al., Plant Cell 20:2763-82 (2008); Zhong et al., Plant Cell 19:2776-92 (2007); Ko et al., Plant J. 60:649-65 (2009); and McCarthy et al., Plant Cell Physiol. 50:1950-64 (2009)). Illustrative examples of gene and protein sequences and/or accession numbers for NST1, NST2, NST3, SND2, SND3, MYB103, MBY85, MYB46, MYB83, MYB58, and MYB63 are provided in PCT/US2012/023182, hereby incorporated by reference.

In some embodiments, the polynucleotide encoding the transcription factor that regulates secondary cell wall production is operably linked to a promoter that is a downstream target of the transcription factor. Similarly, the NST nucleic acid sequence is also linked to a promoter that is a downstream target of the transcription factor. The promoter may be the same promoter or different promoters. In such an embodiment, a promoter is suitable for use with the transcription factor that regulates secondary cell wall production if expression of the promoter is induced, directly or indirectly, by the transcription factor to be expressed, and if the promoter is expressed in the desired location, e.g., the stem of the plant.

### Additional Embodiments for Expressing NST

In another embodiment, the NST polynucleotide is expressed through a transposable element. This allows for constitutive, yet periodic and infrequent expression of the constitutively active polypeptide. The invention also provides for use of tissue-specific promoters derived from viruses including, e.g., the tobamovirus subgenomic promoter (Kumagai, Proc. Natl. Acad. Sci. USA 92:1679-1683, 1995); the rice tungro bacilliform virus (RTBV), which replicates only in phloem cells in infected rice plants, with its promoter which drives strong phloem-specific reporter gene expression; the cassava vein mosaic virus (CVMV) promoter, with highest activity in vascular elements, in leaf mesophyll cells, and in root tips (Verdaguer, Plant Mol. Biol. 31:1129-1139, 1996).

A vector comprising NST nucleic acid sequences will typically comprise a marker gene that confers a selectable phenotype on the cell to which it is introduced. Such markers are known. For example, the marker may encode antibiotic resistance, such as resistance to kanamycin, G418, bleomycin, hygromycin, and the like.

NST nucleic acid sequences of the invention are expressed recombinantly in plant cells as described. As appreciated by one of skill in the art, expression constructs can be designed taking into account such properties as codon usage frequencies of the plant in which the NST nucleic acid is to be expressed. Codon usage frequencies can be tabulated using known methods (see, e.g., Nakamura et al. Nucl. Acids Res. 28:292, 2000). Codon usage frequency tables are available in the art (e.g., from the Codon Usage Database at the internet site www.kazusa.or.jp/codon/.)

When two or more of NSTs (or a NST in conjunction with a sugar synthase) are expressed in combination, they can be expressed from individual promoters. In some embodiments, two or more proteins are expressed from a single promoter, e.g., by incorporating a 2A domain between the two coding sequences.

Additional sequence modifications may be made that are also known to enhance gene expression in a plant. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence may also be modified to avoid predicted hairpin secondary mRNA structures.

### Production of Modified Cells or Transgenic Plants

In some embodiments, the modified eukaryotic cell is a plant, animal or fungal cell. In some embodiments, the animal cell is a mammalian cell. In some embodiments, the fungal cell is a yeast cell. In some embodiments, the yeast cell is a Saccharomyces sp. cell, including but not limited to Saccharomyces cerevisiae. Techniques for genetically modifying eukaryotic cells, such as plant, animal and fungal cells are well known to those skilled in the art. For example, U.S. Provisional Patent Application Ser. No. 61/676,811 teaches such methods for yeast.

In some embodiments, the plant is a grass plant. In some embodiments, the plant of plant cell is Arabidopsis, poplar, eucalyptus, rice, corn, cotton, switchgrass, sorghum, millet, miscanthus, sugarcane, pine, alfalfa, wheat, soy, barley, turfgrass, tobacco, hemp, bamboo, rape, sunflower, willow, or Brachypodium

The present invention provides for transgenic plants comprising recombinant expression cassettes either for expressing heterologous NST. It should be recognized that the term “transgenic plants” as used here encompasses the plant or plant cell in which the expression cassette is introduced as well as progeny of such plants or plant cells that contain the expression cassette, including the progeny that have the expression cassette stably integrated in a chromosome.

Once an expression cassette comprising a polynucleotide encoding a NST (or a polynucleotide sequence designed to suppress or inhibit NST expression as described below) has been constructed, standard techniques may be used to introduce the polynucleotide into a plant in order to modify gene expression. See, e.g., protocols described in Ammirato et al. (1984) Handbook of Plant Cell Culture—Crop Species. Macmillan Publ. Co. Shimamoto et al. (1989) Nature 338:274-276; Fromm et al. (1990) Bio/Technology 8:833-839; and Vasil et al. (1990) Bio/Technology 8:429-434.

Transformation and regeneration of plants is known in the art, and the selection of the most appropriate transformation technique will be determined by the practitioner. Suitable methods may include, but are not limited to: electroporation of plant protoplasts; liposome-mediated transformation; polyethylene glycol (PEG) mediated transformation; transformation using viruses; micro-injection of plant cells; micro-projectile bombardment of plant cells; vacuum infiltration; and Agrobacterium tumeficiens mediated transformation. Transformation means introducing a nucleotide sequence in a plant in a manner to cause stable or transient expression of the sequence. Examples of these methods in various plants include: U.S. Pat. Nos. 5,571,706; 5,677,175; 5,510,471; 5,750,386; 5,597,945; 5,589,615; 5,750,871; 5,268,526; 5,780,708; 5,538,880; 5,773,269; 5,736,369 and 5,610,042.

Transformed plant cells derived by any of the above transformation techniques can be cultured to regenerate a whole plant that possesses the transformed genotype and thus the desired phenotype such as enhanced drought-resistance. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally, e.g., in Klee et al. Ann. Rev. of Plant Phys. 38:467-486, 1987.

One of skill will recognize that after the expression cassette is stably incorporated in transgenic plants and confirmed to be operable, it can be introduced into other plants by sexual crossing. Any of a number of standard breeding techniques can be used, depending upon the species to be crossed.

The expression constructs of the invention can be used to increase the sugar content of cell walls of essentially any plant. The plant may be a monocotyledonous plant or a dicotyledonous plant. In some embodiments of the invention, the plant is a green field plant. In some embodiments, the plant is a gymnosperm or conifer. Thus, the invention has use over a broad range of plants, including species from the genera Asparagus, Atropa, Avena, Brassica, Cannabis, Citrus, Citrullus, Camelina, Capsicum, Cucumis, Cucurbita, Daucus, Fragaria, Glycine, Gossypium, Helianthus, Heterocallis, Hordeum, Hyoscyamus, Lactuca, Linum, Lolium, Lycopersicon, Malus, Manihot, Majorana, Medicago, Nicotiana, Oryza, Panieum, Pannesetum, Persea, Pisum, Pyrus, Prunus, Raphanus, Secale, Senecio, Sinapis, Solanum, Sorghum, Trigonella, Triticum, Vitis, Vigna, and, Zea. In some embodiments, the plant is corn, switchgrass, sorghum, miscanthus, sugarcane, poplar, pine, wheat, rice, soy, cotton, barley, turf grass, tobacco, potato, bamboo, rape, sugar beet, sunflower, willow, and eucalyptus. In further embodiments, the plant is reed canarygrass (Phalaris arundinacea), Miscanthus× giganteus, Miscanthus sp., sericea lespedeza (Lespedeza cuneata), millet, ryegrass (Lolium multiflorum, Lolium sp.), timothy, Kochia (Kochia scoparia), forage soybeans, alfalfa, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, or Kentucky bluegrass among others. In some embodiments, the plant is an ornamental plant. In some embodiments, the plant is a grass plant. In some embodiment, the plant is a vegetable- or fruit-producing plant. In some embodiments, the plant is a plant that is suitable for generating biomass, including plants as noted above, e.g., Arabidopsis, poplar, eucalyptus, rice, corn, switchgrass, sorghum, millet, miscanthus, sugarcane, pine, alfalfa, wheat, soy, barley, turfgrass, tobacco, hemp, bamboo, rape, sunflower, willow, Jatropha, and Brachypodium.

In some embodiments, the plant into which the expression construct comprising a nucleic acid sequence that encodes NST (or that is designed to inhibit expression of NST) is introduced is the same species of plant from which the NST sequence, and/or the promoter driving expression of the NST sequence, is obtained. In some embodiments, the plant into which the expression construct is introduced is a different species of plant compared to the species from which the NST and/or promoter sequence was obtained.

Plants that overexpress NST can be identified using any known assay, including analysis of RNA, protein, or sugar composition. With respect to this aspect of the invention, the plants have enhanced sugar levels, wherein the sugar corresponds to the nucleotide sugar capable of being transported by the NST. The sugar levels can be determined directly or indirectly, wherein such methods are well known in the art.

### Modification of Plants to Decrease Sugar Production

In one aspect, the invention also provides a plant in which expression of NST is inhibited, thereby resulting in reduced levels of the sugar in the plant. In some embodiments, the plant is modified to have a level of NST activity that is reduced throughout the entire plant. In some embodiments, the plant is modified to reduce NST activity in a subset of cells or tissues of the plant. The genetic background of the plant can be modified according to any method known in the art, such as antisense, siRNA, microRNA, dsRNA, sense suppression, mutagenesis, or use of a dominant negative inhibition strategy. In some embodiments, the level of expression of the protein is reduced.

### Gene Silencing Techniques

In some embodiments, expression of a NST is inhibited by an antisense oligonucleotide. In antisense technology, a nucleic acid segment from the desired gene is cloned and operably linked to a promoter such that the antisense strand of RNA will be transcribed. The expression cassette is then transformed into plants and the antisense strand of RNA is produced. In plant cells, it has been suggested that antisense RNA inhibits gene expression by preventing the accumulation of mRNA which encodes the enzyme of interest, see, e.g., Sheehy et al., Proc. Nat. Acad. Sci. USA, 85:8805-8809 (1988); Pnueli et al., The Plant Cell 6:175-186 (1994); and Hiatt et al., U.S. Pat. No. 4,801,340.

The antisense nucleic acid sequence transformed into plants will be substantially identical to at least a portion of the endogenous gene or genes to be repressed. The sequence, however, does not have to be perfectly identical to inhibit expression. Thus, an antisense or sense nucleic acid molecule encoding only a portion of a NST-encoding sequence can be useful for producing a plant in which expression of NST is inhibited. For antisense suppression, the introduced sequence also need not be full length relative to either the primary transcription product or fully processed mRNA. Generally, higher homology can be used to compensate for the use of a shorter sequence. Furthermore, the introduced sequence need not have the same intron or exon pattern, and homology of non-coding segments may be equally effective. In some embodiments, a sequence of at least, e.g., 20, 25, 30, 50, 100, 200, or more continuous nucleotides (up to mRNA full length) substantially identical to a NST mRNA, or a complement thereof, can be used.

Catalytic RNA molecules or ribozymes can also be used to inhibit expression of a gene encoding a NST polypeptide. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA-cleaving activity upon them, thereby increasing the activity of the constructs.

A number of classes of ribozymes have been identified. One class of ribozymes is derived from a number of small circular RNAs that are capable of self-cleavage and replication in plants. The RNAs replicate either alone (viroid RNAs) or with a helper virus (satellite RNAs). Examples include RNAs from avocado sunblotch viroid and the satellite RNAs from tobacco ringspot virus, lucerne transient streak virus, velvet tobacco mottle virus, solanum nodiflorum mottle virus and subterranean clover mottle virus. The design and use of target RNA-specific ribozymes is described in Haseloff et al. Nature, 334:585-591 (1988).

Another method by which expression of a gene encoding a NST polypeptide can be inhibited is by sense suppression (also known as co-suppression). Introduction of expression cassettes in which a nucleic acid is configured in the sense orientation with respect to the promoter has been shown to be an effective means by which to block the transcription of target genes. For an example of the use of this method to modulate expression of endogenous genes, see Napoli et al., The Plant Cell 2:279-289 (1990); Flavell, Proc. Natl. Acad. Sci., USA 91:3490-3496 (1994); Kooter and Mol, Current Opin. Biol. 4:166-171 (1993); and U.S. Pat. Nos. 5,034,323, 5,231,020, and 5,283,184.

Generally, where inhibition of expression is desired, some transcription of the introduced sequence occurs. The effect may occur where the introduced sequence contains no coding sequence per se, but only intron or untranslated sequences homologous to sequences present in the primary transcript of the endogenous sequence. The introduced sequence generally will be substantially identical to the endogenous NST sequence intended to be repressed. This minimal identity will typically be greater than about 65%, but a higher identity can exert a more effective repression of expression of the endogenous sequences. In some embodiments, sequences with substantially greater identity are used, e.g., at least about 80%, at least about 95%, or 100% identity are used. As with antisense regulation, further discussed below, the effect can be designed and tested to apply to any other proteins within a similar family of genes exhibiting homology or substantial homology.

For sense suppression, the introduced sequence in the expression cassette, needing less than absolute identity, also need not be full length, relative to either the primary transcription product or fully processed mRNA. Furthermore, the introduced sequence need not have the same intron or exon pattern, and identity of non-coding segments will be equally effective. In some embodiments, a sequence of the size ranges noted above for antisense regulation is used, i.e., 30-40, or at least about 20, 50, 100, 200, 500 or more nucleotides.

Endogenous gene expression may also be suppressed by means of RNA interference (RNAi) (and indeed co-suppression can be considered a type of RNAi), which uses a double-stranded RNA having a sequence identical or similar to the sequence of the target gene. RNAi is the phenomenon in which when a double-stranded RNA having a sequence identical or similar to that of the target gene is introduced into a cell, the expressions of both the inserted exogenous gene and target endogenous gene are suppressed. The double-stranded RNA may be formed from two separate complementary RNAs or may be a single RNA with internally complementary sequences that form a double-stranded RNA. Although complete details of the mechanism of RNAi are still unknown, it is considered that the introduced double-stranded RNA is initially cleaved into small fragments, which then serve as indexes of the target gene in some manner, thereby degrading the target gene. RNAi is known to be also effective in plants (see, e.g., Chuang, C. F. & Meyerowitz, E. M., Proc. Natl. Acad. Sci. USA 97: 4985 (2000); Waterhouse et al., Proc. Natl. Acad. Sci. USA 95:13959-13964 (1998); Tabara et al. Science 282:430-431 (1998); Matthew, Comp Funct Genom 5: 240-244 (2004); Lu, et al., Nucleic Acids Res. 32(21):e171 (2004)).

Thus, in some embodiments, inhibition of a gene encoding a NST polypeptide is accomplished using RNAi techniques. For example, to achieve suppression of the expression of a DNA encoding a protein using RNAi, a double-stranded RNA having the sequence of a DNA encoding the protein, or a substantially similar sequence thereof (including those engineered not to translate the protein) or fragment thereof, is introduced into a plant of interest. As used herein, RNAi and dsRNA both refer to gene-specific silencing that is induced by the introduction of a double-stranded RNA molecule, see e.g., U.S. Pat. Nos. 6,506,559 and 6,573,099, and includes reference to a molecule that has a region that is double-stranded, e.g., a short hairpin RNA molecule. The resulting plants may then be screened for a phenotype associated with the target protein, for example, screening for an increase in the extractability of sugar from the plants as compared to wild-type plants, and/or by monitoring steady-state RNA levels for transcripts encoding the protein. Although the genes used for RNAi need not be completely identical to the target gene, they may be at least 70%, 80%, 90%, 95% or more identical to the target gene sequence. See, e.g., U.S,. Patent Publication No. 2004/0029283. The constructs encoding an RNA molecule with a stem-loop structure that is unrelated to the target gene and that is positioned distally to a sequence specific for the gene of interest may also be used to inhibit target gene expression. See, e.g., U.S. Patent Publication No. 2003/0221211.

The RNAi polynucleotides may encompass the full-length target RNA or may correspond to a fragment of the target RNA. In some cases, the fragment will have fewer than 100, 200, 300, 400, 500 600, 700, 800, 900 or 1,000 nucleotides corresponding to the target sequence. In addition, in some embodiments, these fragments are at least, e.g., 50, 100, 150, 200, or more nucleotides in length. In some cases, fragments for use in RNAi will be at least substantially similar to regions of a target protein that do not occur in other proteins in the organism or may be selected to have as little similarity to other organism transcripts as possible, e.g., selected by comparison to sequences in analyzing publicly-available sequence databases.

Expression vectors that continually express siRNA in transiently- and stably-transfected have been engineered to express small hairpin RNAs, which get processed in vivo into siRNAs molecules capable of carrying out gene-specific silencing (Brummelkamp et al., Science 296:550-553 (2002), and Paddison, et al., Genes & Dev. 16:948-958 (2002)). Post-transcriptional gene silencing by double-stranded RNA is discussed in further detail by Hammond et al. Nature Rev Gen 2: 110-119 (2001), Fire et al. Nature 391: 806-811 (1998) and Timmons and Fire Nature 395: 854 (1998).

Yet another way to suppress expression of an endogenous NST gene is by recombinant expression of a microRNA that suppresses a target NST. Artificial microRNAs are single-stranded RNAs (e.g., between 18-25-mers, generally 21-mers), that are not normally found in plants and that are processed from endogenous miRNA precursors. Their sequences are designed according to the determinants of plant miRNA target selection, such that the artificial microRNA specifically silences its intended target gene(s) and are generally described in Schwab et al, The Plant Cell 18:1121-1133 (2006) as well as the internet-based methods of designing such microRNAs as described therein. See also, US Patent Publication No. 2008/0313773.

Another example of a method to reduce levels of NST employs riboswitch techniques (see, e.g., U.S. Patent Application Publication Nos. US20100286082, and US20110245326). Plants having mutant backgrounds

In some embodiments, the level of expression of NST is reduced by generating a plant that has a mutation in a gene encoding the NST. One method for abolishing or decreasing the expression of a gene encoding NST is by insertion mutagenesis using the T-DNA of Agrobacterium tumefaciens. After generating the insertion mutants, the mutants can be screened to identify those containing the insertion in the gene of interest. Mutants containing a single mutation event at the desired gene may be crossed to generate homozygous plants for the mutation (Koncz et al. (1992) Methods in Arabidopsis Research. World Scientific).

Alternatively, random mutagenesis approaches may be used to generate new alleles that will generate truncated or defective (non-functional or poorly active) enzymes or unstable RNA, or to disrupt or “knock-out” the expression of a gene encoding a NST enzyme using either chemical or insertional mutagenesis or irradiation. For example, a procedure known as TILLING (see, e.g. Colbert et al., Plant Physiol 126:480-484, 2001; McCallum et al., Nature Biotechnology 18:455-457, 2000). may be used. In this method, mutations are induced in the seed of a plant of interest. The resulting plants are grown and self-fertilized, and the progeny are assessed, e.g., by PCR, to identify whether a mutated plant has a mutation in the gene of interest, or by evaluating whether the plant has reduced galactan content in a part of the plant that expressed the gene of interest.

An expression cassette comprising a polynucleotide encoding the NST, or transcription factor regulating the production of secondary cell wall and operably linked to a promoter, as described herein, can be expressed in various kinds of plants. The plant may be a monocotyledonous plant or a dicotyledonous plant. In some embodiments of the invention, the plant is a green field plant. In some embodiments, the plant is a gymnosperm or conifer.

In some embodiments, the plant is a plant that is suitable for generating biomass. Examples of suitable plants include, but are not limited to, Arabidopsis, poplar, eucalyptus, rice, corn, switchgrass, sorghum, millet, miscanthus, sugarcane, pine, alfalfa, wheat, soy, barley, turfgrass, tobacco, hemp, bamboo, rape, sunflower, willow, Jatropha, and Brachypodium.

In some embodiments, the plant into which the expression cassette is introduced is the same species of plant as the promoter and/or as the polynucleotide encoding NST or transcription factor (e.g., a vessel-specific promoter, NST, and/or transcription factor from Arabidopsis is expressed in an Arabidopsis plant). In some embodiments, the plant into which the expression cassette is introduced is a different species of plant than the promoter and/or than the polynucleotide encoding NST or transcription factor (e.g., a vessel-specific promoter, NST enzyme, and/or transcription factor from Arabidopsis is expressed in a poplar plant). See, e.g., McCarthy et al., Plant Cell Physiol. 51:1084-90 (2010); and Zhong et al., Plant Physiol. 152:1044-55 (2010).

### Methods of Using Plants Having Modified NST Expression

Plants, parts of plants, or plant biomass material from plants having modified NST expression can be used for a variety of purposes. In embodiments, in which NST is overexpressed, the plants, parts of plants, or plant biomass material may be used in a conversion reaction to generate an increased amount of bioenergy as compared to wild-type plants. For example, the plants, parts of plants, or plant biomass material can be used in a saccharification reaction to generate an increased amount of soluble and fermentable sugar compared to wild-type plants. In some embodiments, the plants, parts of plants, or plant biomass material are used to increase biomass yield or simplify downstream processing for wood industries (such as paper, pulping, and construction) as compared to wild-type plants. In some embodiments, the plants, parts of plants, or plant biomass material are used to increase the quality of wood for construction purposes. In some embodiments the plants, or parts of plants are used to improve the quality of textile fiber or simplify the downstream processing for textile industry. In some embodiments the plants, or parts of plants, are used as a raw material for pectin production.

Methods of conversion, for example biomass gasification, are known in the art. Briefly, in gasification plants or plant biomass material (e.g., leaves and stems) are ground into small particles and enter the gasifier along with a controlled amount of air or oxygen and steam. The heat and pressure of the reaction break apart the chemical bonds of the biomass, forming syngas, which is subsequently cleaned to remove impurities such as sulfur, mercury, particulates, and trace materials. Syngas can then be converted to products such as ethanol or other biofuels.

Methods of enzymatic saccharification are also known in the art. Briefly, plants or plant biomass material (e.g., leaves and stems) are optionally pre-treated with hot water, dilute acid, alkali, or ionic liquid followed by enzymatic saccharification using a mixture of cellulases and hemicellulases and pectinases in buffer and incubation of the plants or plant biomass material with the enzymatic mixture. Following incubation, the yield of the saccharification reaction can be readily determined by measuring the amount of reducing sugar released, using a standard method for sugar detection, e.g. the dinitrosalicylic acid method well known to those skilled in the art. Plants engineered in accordance with the invention provide a higher sugar yield as compared to wild-type plants.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Characterization of an Arabidopsis UMP/UDP-Gal, UDP-Rha and UDP-Ara Antiporter Family

The matrix of plant cell walls consists of polysaccharides, which are assembled by lycosyltransferases in the Golgi apparatus. The precursors for polysaccharides are activated sugars that are generally synthesized in the cytosol. To transfer nucleotide sugars into the Golgi lumen a family of nucleotide sugar transporters (NSTs) has evolved. NST activities are investigated by expressing Arabidopsis NST proteins in yeast and reconstituting them into liposomes. The activities are determined with radiolabeled nucleotide sugars and with LC-MS/MS analysis of nucleotide sugar uptake. The Golgi-localized NST UDP-GalT2 is not only capable of transporting UDP-Gal but also UDP-Rha and to a lesser extent UDP-Ara in vitro. Transport activity is strictly dependent on counter-transport of UMP. The same transport activities could be observed for two homologous NSTs. While similar, the three transporters differ in the relative preference for the three substrates. Mutant cell wall monosaccharide analysis confirms the biological function of UDP-GalT2 in plants. galt2-1 and galt2-2 mutants have significantly reduced levels of galactose in Arabidopsis leaves whereas overexpression of GalT2 results in an increase of up to 50%.

### The Phosphate Translocator (TP) Gene Family

In the past decade, very few plant NSTs have been functionally characterized at the molecular level. Notably, plant NSTs that have been characterized only account for the transport of GDP-Man, UDP-Gal, UDP-Glc and CMP-sialic acid, although the latter has not been shown to be part of any plant cell wall polymer. Since this collection of nucleotide sugars only represents a small number of GT substrates, there must be additional NSTs mediating the transport of other key nucleotide sugars, such as UDP-Rha, UDP-GlcA, GDP-Fuc, UDP-Xyl and UDP-Ara, which are crucial for proper cell wall biosynthesis. Based on amino acid sequence similarities 52 proteins have been assigned to the PT family with 44 predicted to be involved in nucleotide sugar transport (NST) in the model plant Arabidopsis thaliana (Col-0) (Table 1).

TABLE 1
The phosphate translocator (TP) family from Arabidopsis. Gene
codes as defined as per The Arabidopsis Information Resource
(website for arabidopsis.org/). Activity indicates experimentally
determined substrate using methods outlined below.

| | | | Activity | |
|---|---|---|---|---|
| | AGI code | Alias | major | minor |
| 1 | AT1G12500 | | n.d. | UDP-GalA, |
| | | | | UDP-GlcA |
| 2 | AT3G10290 | | n.d. | n.d. |
| 3 | AT5G04160 | UTR10 | n.d. | n.d. |
| 4 | AT3G11320 | | n.d. | UDP-GalA, |
| | | | | UDP-GlcA |
| 5 | AT5G05820 | | n.d. | n.d. |
| 6 | AT5G55950 | | n.d. | n.d. |
| 7 | AT5G57100 | | n.d. | n.d. |
| 8 | AT1G06890 | | UDP-Xyl | UDP-Arap |
| 9 | AT2G30460 | | UDP-Xyl | UDP-Arap |
| 10 | AT2G28315 | | UDP-Xyl | UDP-Arap/ |
| | | | | UDP-Araf |
| 11 | AT1G21070 | UTR8 | UDP-Gal, UDP-Rha | UDP-Arap |
| 12 | AT1G76670 | GALT2 | UDP-Gal, UDP-Rha | UDP-Arap |
| 13 | AT5G42420 | | UDP-Gal, UDP-Rha | UDP-Arap |
| 14 | AT4G39390 | NST-KT1 | UDP-Rha, UDP-Gal | UDP-Arap |
| 15 | AT1G34020 | | UDP-Rha, UDP-Gal | n.d. |
| 16 | AT4G09810 | | UDP-Rha, UDP-Gal | n.d. |
| 17 | AT4G31600 | UTR7/ | n.d. | n.d. |
| | | NST15 | | |
| 18 | AT2G43240 | | n.d. | n.d. |
| 19 | AT3G59360 | NST20 | n.d. | n.d. |
| 20 | AT4G35335 | | n.d. | n.d. |
| 21 | AT5G41760 | | n.d. | n.d. |
| 22 | AT5G65000 | | n.d. | n.d. |
| 23 | AT1G12600 | | n.d. | n.d. |
| 24 | AT4G23010 | UTR2 | n.d. | n.d. |
| 25 | AT1G14360 | | n.d. | n.d. |
| 26 | AT2G02810 | UTR1 | | |
| 27 | AT3G46180 | | | |
| 28 | AT5G59740 | | | |
| 29 | AT1G53660 | | n.d. | n.d. |
| 30 | AT3G14410 | | n.d. | n.d. |
| 31 | AT1G48230 | | UDP-Araf | UDP-Xyl, |
| | | | | UDP-Arap |
| 32 | AT3G17430 | NST19 | UDP-Araf | UDP-Xyl, |
| | | | | UDP-Arap |
| 33 | AT2G25520 | | | |
| 34 | AT4G32390 | NST18 | UDP-Araf | n.d. |
| 35 | AT5G11230 | | | |
| 36 | AT5G25400 | | UDP-Araf | n.d. |
| 37 | AT4G32272 | | n.d. | UDP-GlcNAc |
| 38 | AT1G07290 | GONST2 | | |
| 39 | AT2G13650 | GONST1 | GDP-Glc, GDP-Man, | n.d. |
| | | | GDP-Fuc | |
| 40 | AT1G76340 | GONST3 | | |
| 41 | AT5G19980 | GONST4 | n.d. | n.d. |
| 42 | AT1G06470 | | n.d. | n.d. |
| 43 | AT1G21870 | GONST5 | n.d. | n.d. |
| 44 | AT1G77610 | GALT1 | n.d. | n.d. |

### Determination of Transporter Activity of Arabidopsis NSTs

Each NST is cloned and heterologously expressed in yeast, reconstituted yeast microsomal proteins are incorporated into liposomes and transport assays are performed. Essentially, liposomes are loaded with either GMP or UMP and incubated with a collection of 10 nucleotide sugars (UDP-Glc, UDP-Gal, UDP-GlcA, UDP-GalA, UDP-Rha, UDP-Arap, UDP-Araf, GDP-Man, GDP-Fuc, UDP-GlcNAc) for up to 1 hour. Potential nucleotide sugars that have not yet been not tested comprise GDP-Glc, UDP-Api, ADP-Glc, GDP-Gal, UDP-GalNAc, CMP-Kdo, CMP-DHA, CMP-Sialic acid (not in plants) and the like. Incubated liposomes are washed and transported nucleotide sugars profiled using LC-MS/MS. Washed liposomes are and re-suspended in a buffer and nucleotide sugar separations undertaken using porous graphitic carbon as the stationary phase (Hypercarb) using a recently developed approach. The detection of nucleotide sugars separated by porous graphitic carbon is undertaken on a 4000 QTRAP® LC/MS/MS system equipped with a TurbolonSpray® ion source. Specific compound-dependent MS parameters for each nucleotide sugar are determined by direct infusion into the MS of individual standards dissolved in 50% acetonitrile (concentration of 1 pmol/μL) at a flow rate of 20 μL/min. The 4000 QTRAP® is operated in negative ion mode using the multiple reaction monitoring (MRM) scan type. The abundance of separated nucleotide sugars (transporter preference) are determined using MultiQuant™ 2.1 software by integrating the signal peak area.

### The Role of GalT2 in Planta

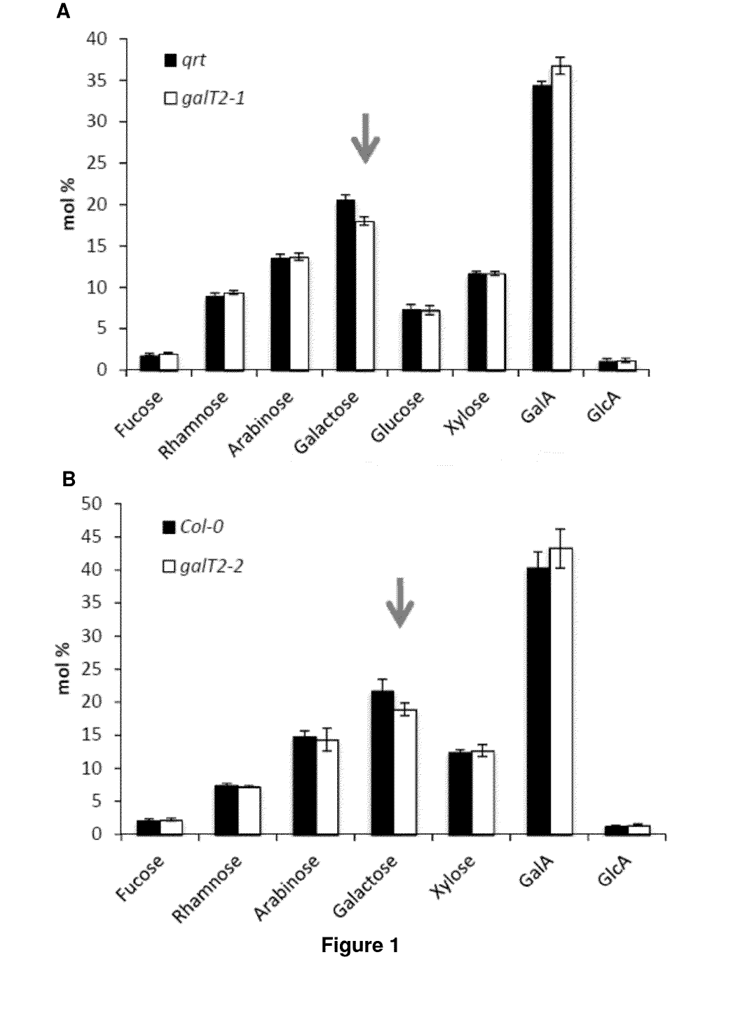

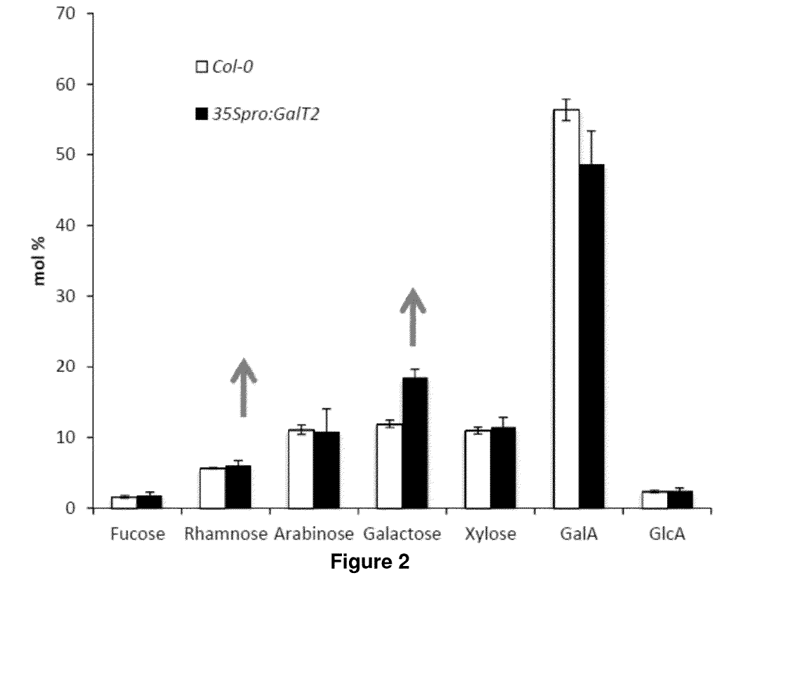

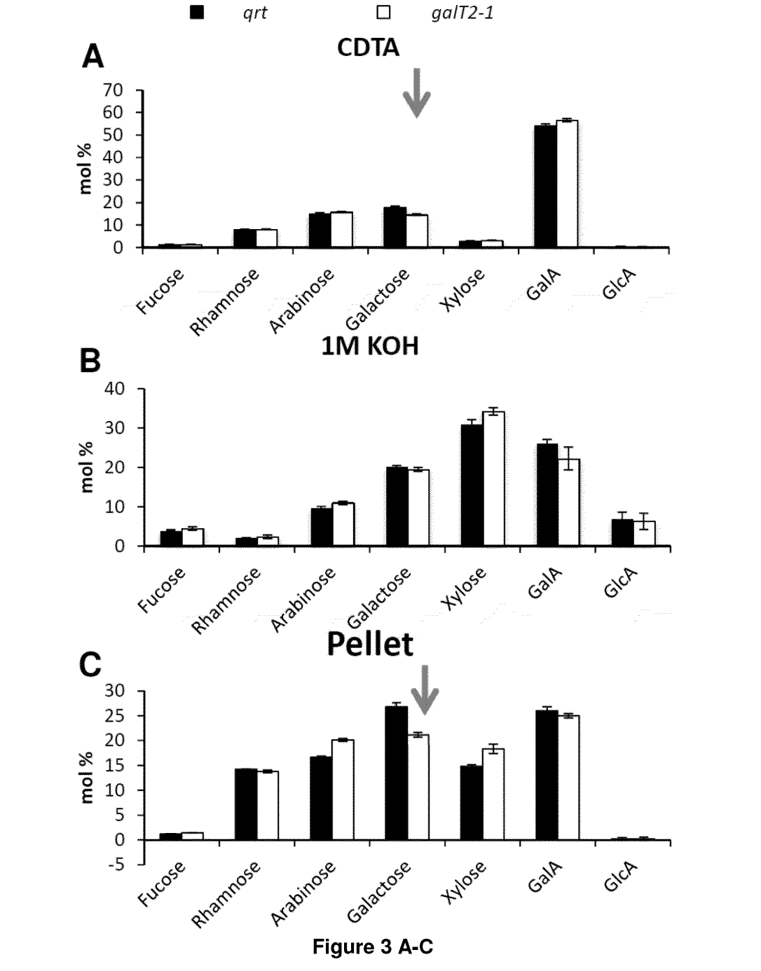

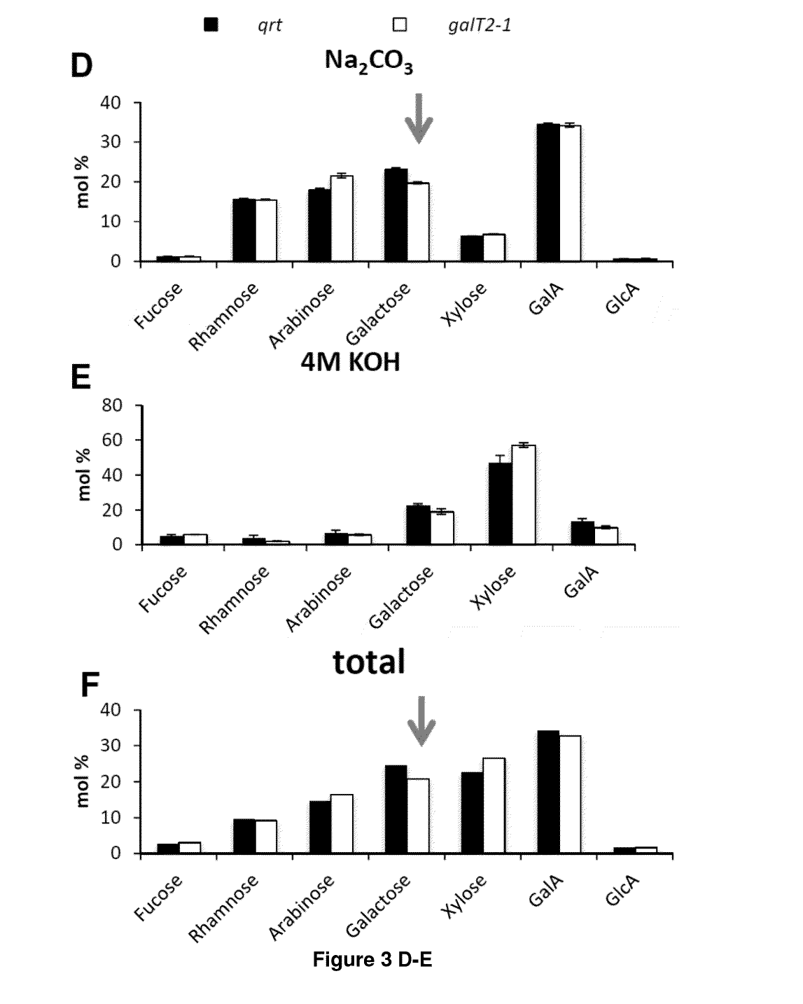

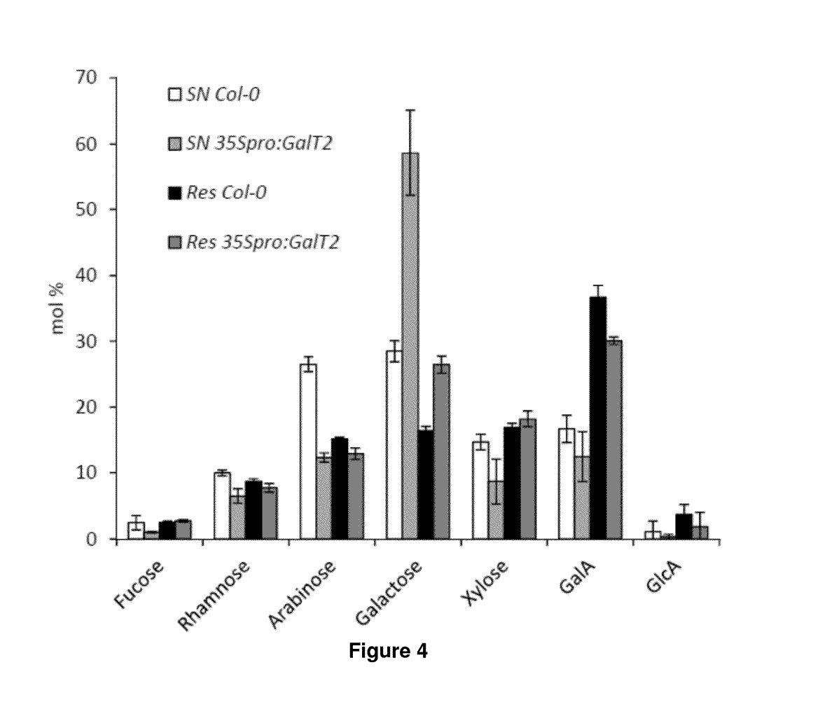

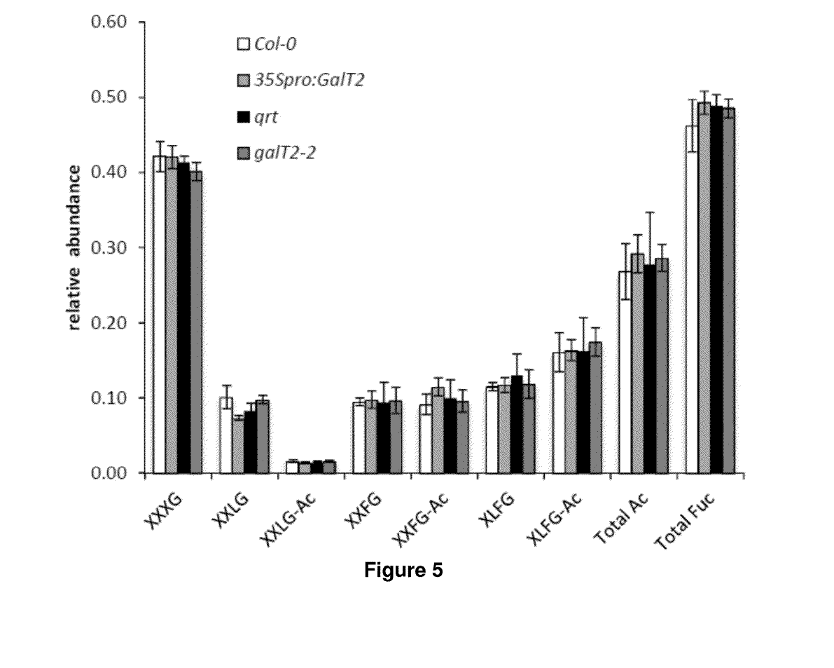

In order to assess the in vivo function of some of these NSTs, the GalT2 mutant and an overexpressor line are analyzed for morphological changes as well as cell wall monosaccharide composition. galT2 mutants show a significant reduction of 13% in galactose content and a resultant increase in galacturonic acid of the total cell wall whereas other monosaccharides, including rhamnose, are unchanged (FIG. 1). When GalT2 is overexpressed under the control of the 35S promoter plants accumulated galactose in leaves of up to 50% in certain lines (FIG. 2). However the reduction or increase in galactose does not affect the morphology of the plants compared to the wild type. To examine what polymers are primarily affected by the change in galactose content in galt2 mutants cell walls are sequentially extracted with CDTA, Na₂CO₃ and KOH. The results indicate that the major change in galactose content is in pectin-rich fractions rather than in the hemicellulose-rich KOH fractions (FIG. 3). In agreement with this, most of the accumulating galactose in GALT2 overexpressing plants is found in a fraction that could be digested with beta-1,4-galactanase (FIG. 4). The main hemicellulose in Arabidopsis primary walls is xyloglucan. To test if xyloglucan is altered in mutant and 35Spro:GalT2-YFP plants enzymatic fingerprinting is performed using a xyloglucanase and MALD-TOF. However no significant difference is revealed when compared to the wild type, proving that the UDP-Gal transported by GalT2 is not built into xyloglucan (FIG. 5).

### Example 2

Knockout mutants and overexpressing lines of the NSTs depicted in Tables 2 and 3 are constructed. They are analyzed and the following activities of each are determined as indicated.

TABLE 2

| AGI code | Activity |
|---|---|
| At1g12500 | UDP-Gal, UDP-Glc |
| At3g11320 | UDP-Gal, UDP-Glc |
| At3g10290 | UDP-Gal, UDP-Glc |
| At5g04160 | UDP-Gal, UDP-Glc |
| At3g11320 | UDP-Gal, UDP-Glc |
| At1g06890 | UDP-Xyl, UDP-Arap |
| At2g30460 | UDP-Xyl, UDP-Arap |
| At2g28315 | UDP-Xyl, UDP-Arap |
| At1g21070 | UDP-Rha/UDP-Gal |
| At5g42420 | UDP-Rha/UDP-Gal |
| At4g39390 | UDP-Rha/UDP-Gal |
| At1g76670 | UDP-Rha/UDP-Gal |
| At1g34020 | UDP-Rha/UDP-Gal |
| At4g09810 | UDP-Rha/UDP-Gal |
| At1g48230 | UDP-Araf minor UDP-Xyl, UDP-Arap |
| At3g17430 | UDP-Araf minor UDP-Xyl, UDP-Arap |
| At2g25520 | UDP-Araf minor UDP-Xyl, UDP-Arap |
| At4g32390 | UDP-Araf minor UDP-Xyl, UDP-Arap |
| At5g11230 | UDP-Araf minor UDP-Xyl, UDP-Arap |
| At5g25400 | UDP-Araf minor UDP-Xyl, UDP-Arap |
| At5g19980 | GDP-Fuc |

TABLE 3

| | | | | Activity | |
|---|---|---|---|---|---|
| | AGI code | Alias | ID | major | minor |
| 1 | AT1G12500 | | TP2 | UDP-GalA, UDP-GlcA | n.d. |
| 2 | AT3G10290 | | NST8 | UDP-GalA, UDP-GlcA | n.d. |
| 3 | AT5G04160 | UTR10 | NST6 | UDP-GalA, UDP-GlcA | n.d. |
| 4 | AT3G11320 | | NST1 | UDP-GalA, UDP-GlcA | n.d. |
| 5 | AT5G05820 | | NST2 | UDP-GalA, UDP-GlcA | n.d. |
| 8 | AT1G06890 | | UXT10 | UDP-Xyl | n.d. |
| 9 | AT2G30460 | | UXT12 | UDP-Xyl | n.d. |
| 10 | AT2G28315 | | UXT11 | UDP-Xyl | UDP-Arap/UDP-Araf |
| 11 | AT1G21070 | | UTGT2 | UDP-Gal, UDP-Rha | UDP-Arap/UDP-Araf |
| 12 | AT1G76670 | GALT2 | URGT1 | UDP-Gal, UDP-Rha | UDP-Arap/UDP-Araf |
| 13 | AT5G42420 | | URGT3 | UDP-Gal, UDP-Rha | UDP-Arap/UDP-Araf |
| 14 | AT4G39390 | NST-KT1 | URGT4 | UDP-Rha, UDP-Gal | UDP-Arap/UDP-Araf |
| 15 | AT1G34020 | | URGT6 | UDP-Rha, UDP-Gal | n.d. |
| 16 | AT4G09810 | | URGT5 | UDP-Rha, UDP-Gal | n.d. |
| 31 | AT1G48230 | | NST24 | UDP-Api | UDP-Araf, UDP-Xyl, UDP-Arap |
| 32 | AT3G17430 | NST19 | D2 | UDP-Api | UDP-Araf, UDP-Xyl, UDP-Arap |
| 33 | AT2G25520 | | NST21 | UDP-Araf | n.d. |
| 34 | AT4G32390 | NST18 | C6 | UDP-Araf | n.d. |
| 35 | AT5G11230 | | NST22 | UDP-Araf | n.d. |
| 36 | AT5G25400 | | NST25 | UDP-Araf | n.d. |
| 37 | AT4G32272 | | NST35 | n.d. | UDP-GlcNAc |
| 39 | AT2G13650 | GONST1 | GONST1 | GDP-Man | GDP-Glc/GDP-Fuc |
| 41 | AT5G19980 | GONST4 | B9 | GDP-Fuc | GDP-Man/GDP-Glc |

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A genetically modified eukaryotic host cell comprising (a) a gene encoding a first nucleotide sugar transporter (NST) operably linked to a promoter, wherein the gene and/or the promoter is heterologous to the cell, and/or (b) a native gene encoding a second NTS is disrupted and/or a promoter of the native gene is disrupted.

2. The cell of claim 1, wherein the first NST is a plant, animal or fungal NST.

3. The cell of claim 1, wherein the cell is a plant, animal or fungal cell.

4. A plant comprising the cell of claim 1, or a progeny thereof.

5. The plant of claim 4, wherein the plant is a grass plant.

6. The plant of claim 4, wherein the plant is Arabidopsis, poplar, eucalyptus, rice, corn, cotton, switchgrass, sorghum, millet, miscanthus, sugarcane, pine, alfalfa, wheat, soy, barley, turfgrass, tobacco, hemp, bamboo, rape, sunflower, willow, or Brachypodium

7. A method of obtaining a modified eukaryotic cell of claim 1, comprising: (a) introducing a nucleotide sugar transporter (NST) operably linked to a promoter into a eukaryotic cell, and (b) optionally culturing the eukaryotic cell.

8. The method of claim 9, wherein the eukaryotic cell is a plant cell.

9. The method of claim 10, wherein the method results in an increase of the amount of a polysaccharide or sugar in a plant comprising the plant cell.

10. The method of claim 10, further comprising (c) optionally growing the cultured plant cell into a plant, (d) optionally collecting the plant material from the plant, and (e) optionally incubating plant material from the plant in a saccharification reaction.
